(19) United States
(12) Patent Application Publication
(10) Pub. No.: US 2021/0282866 A1
(43) Pub. Date: Sep. 16, 2021

(54) IMAGE-GUIDED SURGICAL SYSTEMS WITH AUTOMATED TRAJECTORY GUIDE SYSTEMS AND RELATED DEVICES AND METHODS

(71) Applicant: ClearPoint Neuro, Inc., Irvine, CA (US)

(72) Inventors: Kamal Vij, Chandler, AZ (US); Peter G. Piferi, Orange, CA (US); Maxwell Jerad Daly, Redlands, CA (US)

(21) Appl. No.: 17/185,060

(22) Filed: Feb. 25, 2021

Related U.S. Application Data
(60) Provisional application No. 63/136,405, filed on Jan. 12, 2021.
(60) Provisional application No. 63/086,621, filed on Oct. 2, 2020.
(60) Provisional application No. 62/988,609, filed on Mar. 12, 2020.

(51) Int. Cl. A61B 34/20; G06T 7/20; G01R 33/48; G01R 33/28; G01R 33/54

(52) U.S. Cl. CPC A61B 34/20; G06T 7/20; G01R 33/4818; G01R 33/285; A61B 2090/374; G06T 2207/10088; G06T 2207/30241; A61B 2034/2051; G01R 33/543

(57) ABSTRACT

Surgical systems and methods for an MRI suite include a housing residing in an MRI scanner room of the MRI suite. The housing comprises a plurality of motors coupled to at least one motor driver (in the same or a different housing); and a plurality of elongate cables, one coupled to one of the plurality of motors residing in the same housing or a different housing. The elongate cables comprise MRI compatible material and having a length in a range of about 24 inches and 60 inches. One of the cables couples to one of the motors at a first end portion and a corresponding one actuator of the actuators of a trajectory guide at a second end portion.

### RELATED APPLICATIONS

This application claims the benefit of and priority to U.S. Provisional Application Ser. No. 62/988,609 filed Mar. 12, 2020, U.S. Provisional Application Ser. No. 63/086,621 filed Oct. 2, 2020, and U.S. Provisional Application Ser. No. 63/136,405 filed Jan. 12, 2021, the contents of which are hereby incorporated by reference as if recited in full herein.

### FIELD OF THE INVENTION

The present invention relates generally to image-guided surgical systems with trajectory guides.

### BACKGROUND OF THE INVENTION

MRI suites have a control room with MRI scanner operating components such as an MR scanner control console (scanner workstation), an RF amplifier and control cabinet (typically called the control room) and a separate room or chamber holding a high field magnet in which a patient is placed for an MRI procedure (typically called the scanner room).

At least the MRI scanner room of the MRI suite is enclosed in a Faraday shield (e.g., RF shielding) in order to electrically isolate sensitive MRI radio frequency antennas (also known as coils) and restrict them to transmitting and receiving RF nuclear magnetic resonance signals from the patient under examination. An RF-shielded wall separates the two rooms. A removable portion of the RF-shielded wall is typically called a penetration panel. RF shielding is important because it isolates the MRI scanner from external RF sources that can cause artifacts in the MRI image. For a typical MRI scanner room, the RF shielding causes at least 100 dB of signal attenuation of signals in the frequency range of 1 Hz to 150 MHz. Holes or openings made in this shielding can compromise the shielding effectiveness.

In most MRI suites, a grounded waveguide may provide the only access path/space for non-metallic cables, tubes, water or gas lines, and the like between the control room and the Scanner room. All other electrical cables have feed-through filters that are built on the penetration panel which is electrically connected to the Faraday shield. The waveguide is typically a tubular pipe made of copper or brass that extends out a certain distance on each side of the penetration panel. Waveguide depth and diameter is based on the fact that an electromagnetic field attenuates rapidly down a small diameter hole of sufficient depth, provided certain conditions are met. Thus, the diameter and length of the waveguide are chosen to inhibit or prevent RF waves from passing through it. Removable filter boxes have also been used to help avoid permanent modification to MRI suites, see, e.g., co-assigned PCT/US2012/037334. The contents of the patent applications cited above are hereby incorporated by reference as if recited in their entirety herein.

In the past, the CLEARPOINT® surgical system by Clearpoint Neuro, Inc., Irvine, Calif., required a user in the MR scanner room to provide manual input to rotate thumbwheels on a trajectory guide to adjust a trajectory to a target site, such as an intra-brain site, based on suggested calculated turns provided by an image-guided surgical system having a computer control system in the control room using images of different imaging planes to locate and orient a targeting cannula position in three dimensional image space. See, e.g., U.S. Pat. No. 8,315,689, the contents of which are hereby incorporated by reference as if recited in full herein.

### SUMMARY OF EMBODIMENTS OF THE INVENTION

Embodiments of the invention provide surgical systems that obtain images of a trajectory guide in three dimensional image space and remotely, automatically control motors located in an MRI scanner room that are coupled to actuators of the trajectory guide to controllably move one or more of the actuators to position the trajectory guide at an orientation providing a desired intrabody trajectory to thereby provide rapid and accurate alignment of the trajectory guide to the desired trajectory with positional accuracy to the target site.

Embodiments of the invention provide surgical systems having bi-directional communication between a microcontroller in the MR scanner room and a computer system in the MR control room to control motors using the computer system and the microcontroller and to provide status updates of when completion of directional movement of one or more motors has occurred, with the motors being provided directional commands based on instructions from the computer system and microcontroller.

The actuators can comprise a plurality of thumb wheels, one for each of a pitch direction actuator and a roll direction actuator and optionally also an X direction actuator and a Y direction actuator,

The systems can include a plurality of MRI compatible motors, at least one for each of the actuators, in an MRI scanner room.

The motors can be stepper motors coupled to one or more motor drivers.

The surgical systems can include an RF shielded housing enclosing a microcontroller, stepper motors and a motor driver. The housing can be coupled to the bed of an MR scanner to move as a unit therewith to retract and extend with a patient (inside and out of a bore of the magnet).

Embodiments of the invention are directed to image-guided surgical methods. The methods include: providing an image guided surgical system comprising a computer system configured with a surgical procedure workflow; providing a trajectory guide adapted to be held adjacent to or affixed to a patient, the trajectory guide including a plurality of actuators for positionally adjusting an intrabody trajectory defined by the trajectory guide for placing at least one surgical device along a desired intrabody trajectory; providing a plurality of motors and coupling one of the plurality of motors to a corresponding one of the plurality of actuators; and providing at least one housing. The least one housing contains a controller that communicates with the plurality of motors. The methods also include transmitting instructions from the computer system of the image guided surgical system to the controller then to one or more of the plurality of motors to controllably activate one or more of the motors to turn a corresponding one or more of the plurality of actuators to adjust the intrabody trajectory defined by the trajectory guide.

The image guided system can be an MRI-image guided system that is coupled to an MRI scanner system. The computer system can reside at least partially in a control room of an MRI suite. The motors can be MRI-compatible motors. The at least one housing can reside in a scanner room of the MRI suite.

Each MRI-compatible motor of the plurality of MRI-compatible motors can be coupled to and/or include a control cable that extends to one or more of the at least one housing.

The control cable can have a length in a range of about 24 inches and 60 inches. The control cable and the at least one housing can be formed of one or more MRI compatible materials

The plurality of motors can be provided as a plurality of separate motor assemblies. Each of the motor assemblies can have a motor assembly housing having an open channel under a sealed compartment. The sealed compartment can hold a respective one of the plurality of motors. Each of the motor assemblies can also include an actuator connector held in the open channel. The method can include placing a respective actuator of the plurality of actuators into a corresponding open channel and attaching the actuator connector to the respective actuator.

The method can include matching colors of actuators and actuator connectors to identify actuator and actuator connector pairs, then attaching the matching color actuator and actuator connector pairs to couple the plurality of motors to the plurality of actuators.

The at least one housing can have a plurality of motor drivers that are coupled to the plurality of motors. The method can further include controlling the plurality of motors using the motor drivers and transmitting status updates of motor operation of respective motors of the plurality of motors to the computer system of the image guided surgical system.

The computer system of the image guided surgical system can be configured to: transmit slice plane parameters to a computer system of the MRI scanner system; evaluates images obtained by the MRI scanner system using the slice plane parameters; identify a first intrabody trajectory provided by the trajectory guide as a current defined intrabody trajectory; compare the first intrabody trajectory to the desired intrabody trajectory that intersects a target intrabody site; calculate directional adjustments of one or more of the plurality of actuators of the trajectory guide to achieve the desired intrabody trajectory; then transmit the instructions to a controller coupled to motor drivers that are coupled to the MRI-compatible motors, then direct one or more of the plurality of MRI-compatible motors to turn a corresponding actuator to adjust the first intrabody trajectory to a new defined intrabody trajectory to thereby provide the desired intrabody trajectory.

The computer system can be configured to direct two or more of the MRI-compatible motors to serially turn corresponding actuators.

The computer system can be configured to direct at least two of the MRI-compatible motors to concurrently turn at least two of the cables and corresponding actuators.

The method can include transmitting status data from the one or more of the plurality of MRI-compatible motors to the computer system of the image guided surgical system. The method can include triggering the computer system of the image guided surgical system based, at least in part, on the transmitted status data, to communicate with a computer system of the MRI scanner to initiate slice acquisitions to thereby obtain further image slices.

The plurality of actuators can include a roll actuator and a pitch actuator.

The plurality of actuators can include an X direction actuator and a Y direction actuator.

The plurality of motors can be coupled to respective rotary encoders that have a resolution in a range of about 100-300 μrad.

The computer system of the image guided surgical system can: (a) transmit slice plane parameters to a computer system of the MRI scanner system to obtain images of slices extending across only a top end portion of a targeting cannula, then (b) evaluate the obtained images and identifies whether a projected error of alignment relative to the desired intrabody trajectory is within a first defined value. If so, the computer system of the image guided surgical system then (c) transmit slice plane parameters to the computer system of the MRI scanner system to initiate acquisition of at least a first stack of images in a first plane with image slices that extend over at least a major length and/or width of the targeting cannula, and then (d) evaluate the first stack of images and identifies whether a current projected error of alignment relative to the desired intrabody trajectory is within a second defined value. The second defined value can be less than the first defined value.

The MRI-compatible motors are MRI-compatible stepper motors coupled to respective encoders.

The at least one housing has a plurality of motor drivers held therein. The method further includes transmitting direction command instructions to one or more of the plurality of motor drivers that then directs an appropriate one or more of the plurality of motors to turn at a defined speed and time to carry out positional adjustments of one or more of the plurality of actuators.

Yet other embodiments are directed to a surgical system for an MRI suite. The system includes a housing that contains a controller that is configured to reside in an MRI scanner room of the MRI suite. The system also includes a plurality of motor assemblies including at least a first motor assembly housing and a second separate motor assembly housing. The first motor assembly housing encloses a first motor and a first actuator connector. The first actuator connector is coupled to the first motor. The second motor assembly housing encloses a second motor and a second actuator connector. The second actuator connector is coupled to the second motor.

The system can include a trajectory guide comprising at least first and second spaced apart actuators. The first actuator is connected to the first actuator connector of the first motor assembly and the second actuator is coupled to the second actuator connector of the second motor assembly.

The first motor assembly housing has an enclosure cap coupled to an enclosure leg. A motor shaft coupler and a secondary drive shaft as well as the first actuator connector and the first actuator reside in the enclosure leg of the first motor assembly housing. The second motor assembly housing has an enclosure cap coupled to an enclosure leg. A motor shaft coupler and a secondary drive shaft as well as the second actuator connector and the second actuator can all reside in the enclosure leg of the second motor assembly housing.

The first motor can be sealably held in the enclosure cap above the enclosure leg of the first motor assembly housing. The second motor can be sealably held in the enclosure cap above the enclosure leg of the second motor assembly housing. The first motor assembly housing and the second motor assembly housing can be adjacent and each can have a maximal longitudinal length of about 1.5 inches to about 3.5 inches.

The first motor assembly housing can have a first elongate cable extending outward thereof that can be coupled at a first end portion to the first motor and that can be coupled at a second end portion to the housing comprising the controller. The second motor assembly housing can have a second elongate cable extending outward thereof coupled at a first end portion to the second motor and coupled at an opposing second end portion to the housing comprising the controller.

The first and second elongate cables can be formed of MRI compatible material or MRI compatible materials and can have a length in a range of about 24 inches to about 60 inches. The first and second actuator connectors can be configured to releasably couple to a respective thumbwheel of respective first and second actuators of a trajectory guide.

The system can have a scanner bed attachment assembly coupled to or coupleable to the housing comprising the controller.

The housing containing the controller, the first motor assembly housing and the second motor assembly housing can all be non-ferromagnetic.

The housing containing the controller also contains at least one motor driver in communication with the controller and a power supply coupled to the controller and/or the at least one motor driver.

The first and second motors can be coupled to respective rotary encoders that can have a resolution in a range of about 100-300 μrad.

The housing with the controller can have RF shielding.

The housing with the controller can be coupled to at least one fiber optic cable that has a length sufficient to extend through a wave guide of an MRI suite between a control room and a scanner room.

A computer system of an image guided surgical system can be triggered, based, at least in part, on transmitted status data of the first and/or second motor, to communicate with a computer system of an MR scanner system to initiate slice acquisitions to thereby obtain image slices.

An image-guided surgical system. The system includes a computer system configured with a surgical procedure workflow. The computer system resides at least partially in a control room of a magnetic resonance imaging (MRI) suite of a magnetic resonance (MR) scanner system. The image guided surgical system also includes a trajectory guide adapted to be held adjacent to or affixed to a patient. The trajectory guide can have a plurality of actuators for positionally adjusting an intrabody trajectory defined by the trajectory guide for placing at least one surgical device along a desired intrabody trajectory; and a plurality of motors that reside in a scanner room of the MRI suite, one motor in communication with one of the plurality of actuators of the trajectory guide. The motors are MRI compatible motors and instructions from the computer system of the image guided surgical system controllably activates one or more of the motors to turn to adjust the intrabody trajectory defined by the trajectory guide.

The system can further include a drive control enclosure with a controller and motor drivers. The drive control enclosure can reside in the scanner room. The computer system of the image guided surgical system can be further configured to: transmit slice plane parameters to a computer system of the MR scanner system; evaluate images obtained by the MR scanner system using the slice plane parameters; identify a first intrabody trajectory provided by the trajectory guide as a current defined intrabody trajectory; compare the first intrabody trajectory to the desired intrabody trajectory that intersects a target intrabody site; calculate directional adjustments of one or more of the actuators of the trajectory guide to achieve the desired intrabody trajectory; then transmit instructions to the controller in the drive control enclosure which then directs the motor drivers to communicate with one or more of the motors to turn a corresponding actuator to adjust the first intrabody trajectory to a new defined intrabody trajectory.

The drive control enclosure can contain a power supply and the controller and can reside outside a 5 Gauss line in the scanner room of the MRI suite to be at a low Gauss region while coupled to the plurality of motors.

The plurality of motors can be coupled to encoders that provide status information to the controller.

The plurality of motors can be coupled to respective rotary encoders that can have a resolution in a range of about 100-300 μrad.

Each motor of the plurality of motors can be held in a respective motor assembly housing. Each respective motor assembly housing can be spaced apart from another.

The system can include a plurality of motor assembly housings that are spaced apart and project outward from the trajectory guide. One of the plurality of motors is held in a corresponding one of the plurality of motor assembly housings. The plurality of motor assembly housings also contain a respective actuator connector. Each actuator connector can be coupled to a corresponding motor.

The first motor assembly housing can have an enclosure cap coupled to an enclosure leg. The first actuator connector and the first actuator can reside in the enclosure leg of the first motor assembly housing. The second motor assembly housing can have an enclosure cap coupled to an enclosure leg. The second actuator connector and the second actuator can reside in the enclosure leg of the second motor assembly housing.

The plurality of motor assembly housings can be provided as a first motor assembly housing for a pitch actuator connector and a second motor assembly housing for a roll actuator connector. The plurality of motors includes a first motor and a second motor. The first motor assembly housing and the second motor assembly housing have a respective enclosure cap coupled to a corresponding enclosure leg. The first motor can be sealably held in the enclosure cap above the enclosure leg of the first motor assembly housing. The second motor can be sealably held in the enclosure cap above the enclosure leg of the second motor assembly housing. The first motor assembly housing and the second motor assembly housing can be adjacent and can have a maximal longitudinal length of about 1.5 inches to about 3.5 inches.

Yet other embodiments are directed to a surgical motor assembly for an image-guided surgical navigation system. The surgical motor assembly includes: a motor assembly housing: a motor held in the housing; and an actuator connector held in the housing below the motor. The actuator connector is coupled to the motor.

The motor assembly housing can have an enclosure cap coupled to an enclosure leg. The actuator connector can reside in the enclosure leg, and the leg has a wall that surrounds an open downwardly extending channel.

The motor can be sealably held in the enclosure cap above the enclosure leg. The motor assembly housing has a maximal height or longitudinal length of about 1.5 inches to about 3.5 inches.

The assembly can include an elongate cable coupled to the motor and extending outward of the motor assembly housing.

The motor is an MRI-compatible stepper motor. The elongate cable and the motor assembly housing can be formed of an MRI compatible material or MRI compatible materials. The elongate cable can have a length in a range of about 24 inches to about 60 inches. The actuator connector can be sized and configured to releasably couple to a thumbwheel of an actuator of a trajectory guide.

The enclosure leg of the motor assembly housing can have a first segment at an upper portion that is angled that merges into a second segment that extends along a medial and lower portion that is vertical. The first segment can be attached to a bottom of the enclosure cap. The first segment can enclose a first drive shaft coupler and the second segment can enclose a U-joint coupler that is attached to the first drive shaft coupler at an upper end and that is attached to the actuator connector at the second end.

The assembly can include a motor shaft coupler attached to a secondary drive shaft both extending under the motor in the motor assembly housing. The secondary drive shaft can have a diameter that is less than a diameter of the secondary drive shaft coupler. The secondary drive shaft can have a length that extends into the actuator connector and a length that extends into the secondary drive shaft.

A proximal end of the actuator connector can reside closely spaced apart a range of about 0.001 mm and about 10 mm from a distal end of the motor shaft coupler inside the motor assembly housing.

The actuator connector can reside a distance in a range of about 10 mm to about 50 mm above an open end of a channel of an enclosure leg of the motor assembly housing. The channel can be sized and configured to receive an entire thumbwheel of a target actuator that couples to the actuator connector inside the channel.

The channel can be further configured to receive a projecting feature of a trajectory guide holding the target actuator under the thumbwheel.

The surgical systems can include plurality of long cables, optionally comprising torque transfer shafts, coupled at one end to a respective actuator and at another end to a respective stepper motor.

The cables can comprise an internal cable surrounded by an external cable such as a shaft or sleeve.

The internal cable and the external cable can be formed of MRI compatible (non-ferromagnetic) material.

The internal cable can be formed of nitinol steel. The external cable can comprise fiberglass.

Each end of the cable can comprise a connector, one connector configured to couple to an actuator held by the trajectory guide and the other connector configured to (indirectly) couple to a stepper motor, optionally via a shaft coupler.

The stepper motors can be MRI compatible.

The stepper motors can be piezoelectric stepper motors.

The motors can be coupled to respective rotary encoders that have a resolution in a range of about 100-300 gad.

Embodiments of the present invention are directed to image-guided surgical methods. The methods include providing an image guided surgical system comprising a computer system configured with a surgical procedure workflow. The computer system resides at least partially in a control room of an MRI suite. The methods also include providing a trajectory guide adapted to be held adjacent to or affixed to a patient, the trajectory guide comprising a plurality of actuators for positionally adjusting an intrabody trajectory defined by the trajectory guide for placing at least one surgical device along a desired intrabody trajectory and providing at least one housing containing a plurality of motors. The at least one housing resides in a scanner room of the MRI suite. At least one of the at least one housing that contains the plurality of motors is coupled to or coupleable to a bed of an MRI scanner and the motors are MRI compatible motors. The methods also include providing a plurality of elongate cables comprising longitudinally opposing first and second end portions, the first end portion of a respective cable coupled to or coupleable to a corresponding one of the plurality of motors and the second end portion of the respective cable coupled to or coupleable to a corresponding one of the plurality of actuators and transmitting instructions from the computer system of the image guided surgical system to controllably activate one or more of the motors to turn to adjust the intrabody trajectory defined by the trajectory guide.

The cables each have a length in a range of about 24 inches and 60 inches, and s are formed of one or more MRI compatible materials.

The at least one housing can include a first housing holding the motors. The method can further include providing a second housing holding defining a drive control enclosure. The drive control enclosure includes at least one motor driver, typically a plurality of motor drivers, coupled to the motors in the scanner room by a cable assembly. The method can further include controlling the motors using the motor drives in the drive control enclosure and transmitting status updates of motor operation to the computer system of the image guided surgical system.

The cables can include torque transfer shafts with an internal cable with a length and an outer cable fixedly attached to the internal cable. The internal cable can have first and second opposing end portions that extend out of each opposing end of the outer cable. The first end portion can have a first connector and the second end portion can have a second connector. The first connector can be attached to a shaft extending out of a flexible shaft coupler. The flexible shaft coupler can reside adjacent a corresponding motor, optionally external to the at least one housing. The second connector can be attached to a thumbwheel or knob of a respective actuator of the actuators of the trajectory guide.

The at least one housing can further enclose a converter and a communications unit. The computer system of the image guided surgical system can be coupled to a converter in the control room. The method can further include transmitting status updates from the converter in the at least one housing to the converter in the control room, then to the computer system of the image guided surgical system.

The computer system of the image guided surgical system can also be configured to: transmit slice plane parameters to a computer system of an MR scanner system; evaluate images obtained by the MR scanner system using the slice plane parameters; identify a first intrabody trajectory provided by the trajectory guide as a current defined intrabody trajectory; compare the first intrabody trajectory to the desired intrabody trajectory that intersects a target intrabody site; calculate directional adjustments of one or more of the actuators of the trajectory guide to achieve the desired intrabody trajectory; then transmit the instructions to a controller coupled to at least one motor driver which then directs one or more of the motors to turn a corresponding cable to turn a corresponding actuator to adjust the first intrabody trajectory to a new defined intrabody trajectory.

The directing can be carried out to direct two or more of the motors to serially turn respective cables and corresponding actuators.

The directing can be carried out to direct at least two of the motors to concurrently turn at least two of the cables and corresponding actuators.

The method can also include transmitting status data from the one or more of the motors to the computer system of the image guided surgical system.

The method can further include triggering the computer system of the image guided surgical system based, at least in part, on the transmitted status data, to communicate with a computer system of the MRI scanner to initiate slice acquisitions to thereby obtain further image slices.

The actuators can include a roll actuator, a pitch actuator, an X direction actuator and a Y direction actuator.

The computer system of the image guided surgical system can be configured to (a) transmit slice plane parameters to a computer system of the MRI scanner to obtain images of slices extending across only a top end portion of the targeting cannula, then (b) evaluate the obtained images and identifies whether a projected error of alignment relative to the desired intrabody trajectory is within a first defined value. If so, the computer system of the image guided surgical system can then (c) transmit slice plane parameters to the computer system of the MRI scanner to initiate acquisition of at least a first stack of images in a first plane with image slices that extend over at least a major length and/or width of the targeting cannula, then (d) evaluate the first stack of images and identifies whether a current projected error of alignment relative to the desired intrabody trajectory is within a second defined value. The second defined value can be less than the first defined value.

The MRI-compatible motors can be MRI-compatible stepper motors coupled to respective encoders. The MRI-compatible stepper motors and the encoders can be held together in a single housing of the at least one housing.

A controller in a drive motor enclosure that also holds one or more motor drivers can receive the transmitted instructions. The method can further include causing the controller to transmit direction command instructions to one or more motor drivers that then directs an appropriate one or more of the motors to turn at a defined speed and time to carry out positional adjustments of one or more of the actuators.

Other embodiments are directed to surgical systems for an MRI suite. The systems can include at least one housing residing in an MRI scanner room of the MRI suite. The at least one housing a plurality of motors. The systems also include a plurality of elongate cables, one coupled to a corresponding one of the plurality of motors. The elongate cables are formed of MRI compatible material or MRI compatible materials and have a length in a range of about 24 inches to about 60 inches. Each cable of the plurality of cables can indirectly or directly couple to a respective motor of the plurality of motors at a first end portion and couple to a thumbwheel or knob of a corresponding respective actuator of actuators of a trajectory guide at a second end portion.

The systems can further include a scanner bed attachment assembly coupled to or coupleable to the at least one housing.

The plurality of cables can each have a first connector and a second connector. The first connector can be attached to a corresponding one of the plurality of motors. The second connector can be attached to the thumbwheel or knob of a respective actuator of the actuators of the trajectory guide.

The at least one housing can include a first housing and a second housing which are both non-ferromagnetic. The first housing can contain the plurality of motors and the second housing can contain the at least one motor driver, the at least one microcontroller and a power supply. The system can also include a cable extending between the first and second housings that electrically connects the one or more motor drivers to the motors.

The systems can further include a plurality of flexible shaft couplers residing adjacent respective motors of the plurality of motors, each of the flexible shaft couplers comprising longitudinally opposing first and second end portions and a plurality of first shafts that are rigid and extend out of a respective motor of the plurality of motors. Each first shaft can have a first length and an outer end that extends into the first end portion of the flexible shaft coupler. The systems can also include a plurality of second shafts, wherein each second shaft extends out of the second end portion of the flexible shaft coupler. Each second shaft can have a second length that is greater than the first length. Each second shaft can merge into a connector that receives a first connector of a respective cable.

The connector of the second shaft has a knob with an outer diameter that is greater than the second shaft.

The flexible shaft coupler and the second shaft can reside external to the at least one housing.

The at least one housing can have RF shielding.

The at least one housing can be coupled to a fiber optic cable that has a length sufficient to extend through a wave guide of an MRI suite between a control room and a scanner room.

Still other embodiments are directed to image-guided surgical systems. The systems include a computer system configured with a surgical procedure workflow. The computer system resides at least partially in a control room of an MRI suite. The systems include a trajectory guide adapted to be held adjacent to or affixed to a patient, the trajectory guide has a plurality of actuators for positionally adjusting an intrabody trajectory defined by the trajectory guide for placing at least one surgical device along a desired intrabody trajectory and at least one housing containing a plurality of motors, typically provided as single first housing. The at least one housing resides in a scanner room of the MRI suite. At least one of the at least one housing that contains the plurality of motors is coupled to or coupleable to a bed of an MRI scanner. The motors are MRI compatible motors. The systems also include a plurality of elongate cables comprising longitudinally opposing first and second end portions, the first end portion of a respective cable coupled to or coupleable to a corresponding one of the plurality of motors and the second end portion of the respective cable coupled to or coupleable to a corresponding one of the plurality of actuators. Instructions from the computer system of the image guided surgical system controllably activate one or more of the motors to turn to adjust the intrabody trajectory defined by the trajectory guide.

The computer system of the image guided surgical system can be further configured to: transmit slice plane parameters to a computer system of an MR scanner system; evaluate images obtained by the MR scanner system using the slice plane parameters; identify a first intrabody trajectory provided by the trajectory guide as a current defined intrabody trajectory; compare the first intrabody trajectory to the desired intrabody trajectory that intersects a target intrabody site; calculate directional adjustments of one or more of the actuators of the trajectory guide to achieve the desired intrabody trajectory; then transmit instructions to a controller in a drive control enclosure comprising motor drivers which then direct one or more of the motors to turn a corresponding cable to turn a corresponding actuator to adjust the first intrabody trajectory to a new defined intrabody trajectory.

The drive control enclosure can house a power supply and a controller and can be positioned to reside outside a 5 Gauss line to be at a lower Gauss region in the MRI scanner room while coupled to the first housing by a cable assembly.

The motors can be coupled to encoders that can provide status information to the controller in the second housing.

Further features, advantages and details of the present invention will be appreciated by those of ordinary skill in the art from a reading of the figures and the detailed description of the preferred embodiments that follow, such description being merely illustrative of the present invention.

Features described with respect with one embodiment can be incorporated with other embodiments although not specifically discussed therewith. That is, it is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. The foregoing and other aspects of the present invention are explained in detail in the specification set forth below.

### BRIEF DESCRIPTION OF THE DRAWINGS

FIG. 1 is a schematic illustration of an MRI suite with a surgical system having control room components connected to components in an MRI scanner room using one or more fiber optic cables routed through a waveguide in a RF shielded wall according to embodiments of the present invention.

FIG. 2 is a side perspective view of an example trajectory guide of the surgical system shown in FIG. 1.

FIG. 3A is a top perspective view of an example cable useful to connect a motor to an actuator on the trajectory guide of the surgical system according to embodiments of the present invention.

FIG. 3B is an enlarged distal end portion of the cable shown in FIG. 3A.

FIG. 3C is an enlarged proximal end portion of the cable shown in FIG. 3A.

FIG. 4 is a partial side perspective view of a cable connected to an actuator of the trajectory guide shown in FIG. 2.

FIG. 5 is a top view of a portion of components of the surgical system shown in FIG. 1 (with certain components residing in a housing represented by broken line in this drawing).

FIG. 6 is a schematic illustration of example components of the surgical system shown in FIG. 1 that can be provided in a housing residing in the MR scanner room according to embodiments of the present invention.

FIG. 7A is a side perspective view of an example configuration of components in the MRI scanner room for an automated trajectory guide adjustment/control using the surgical system according to embodiments of the present invention.

FIG. 7B is a schematic illustration of another embodiment of an automated trajectory guide/control system with a motor enclosure and a separate drive control enclosure, both configured to reside in the MRI scanner room according to embodiments of the present invention.

FIG. 8 is an example surgical system with bi-directional communication signals providing an automated controlled directional output to the cables to turn actuators of the trajectory guide according to embodiments of the present invention.

FIG. 9 is a schematic illustration of alignment adjustment calculations for aligning a trajectory guide to a desired intrabody trajectory using images of a targeting cannula in 3D image space according to embodiments of the present invention.

FIG. 10 is a schematic illustration of an example server-client configuration of a computer system of an MRI scanner console and a computer system of a workstation providing an image guided surgical system with the computer systems communicating over a local area network (LAN) according to embodiments of the present invention.

FIG. 11 is a flow chart of exemplary operations that can carry out automatically driven alignment adjustments according to embodiments of the present invention.

FIG. 12 is a schematic illustration of a data processing system according to embodiments of the present invention.

FIG. 13A is an exploded, side perspective view of another embodiment of a motor drive system according to embodiments of the present invention.

FIG. 13B is an assembled view of the motor drive system shown in FIG. 13A.

FIG. 14A is an exploded side perspective view of another example embodiment of a motor drive system according to embodiments of the present invention.

FIG. 14B is an assembled view of the motor drive system shown in FIG. 14A.

FIG. 15A is a side perspective view of the motor drive system and actuator shown in FIG. 14A aligned for attachment to a tower of a trajectory guide according to embodiments of the present invention.

FIG. 15B is an enlarged partial side perspective view of the devices shown in FIG. 15A in a pre-attachment configuration.

FIG. 15C is a side perspective assembled view of the devices shown in FIG. 15A.

FIG. 16A is a side perspective view of the motor drive system and actuator shown in FIG. 13A aligned for attachment to a tower of a trajectory guide according to embodiments of the present invention.

FIG. 16B is an enlarged partial side perspective view of the devices shown in FIG. 16A in a pre-attachment configuration.

FIG. 16C is a side perspective assembled view of the devices shown in FIG. 16A.

FIG. 17 is a side perspective view of another embodiment of a motor drive system according to embodiments of the present invention.

FIG. 18A is a side perspective view of the motor drive system shown in FIG. 17 coupled to an actuator of a trajectory guide according to embodiments of the present invention.

FIG. 18B is a top view of the devices shown in FIG. 18A.

### DETAILED DESCRIPTION OF EMBODIMENTS OF THE INVENTION

The present invention now is described more fully hereinafter with reference to the accompanying drawings, in which some embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. The terms “FIG.” and “Fig.” are used interchangeably with the word “Figure” in the specification and/or figures.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

The term “lead” means an electrical path created by one or more wires. The wires are typically insulated wires, particularly where exposed. The term “cable” can refer to an electrical cable, a mechanical cable or a fiber optic cable.

The term “about” means that the noted parameter can vary somewhat from the stated number, typically by +/−20%.

The term “MRI suite” refers to an enclosure that includes at least two rooms or chambers separated by RF shielding and/or an RF shielded wall as is well known to those of skill in the art.

The term “computer system” refers to a device with at least one digital signal processor and/or data processing system. The computer system can be provided as a desktop, laptop, smartphone or other device. A respective computer system can comprise computer program code configured to provide defined operational functions and/or interfaces with other computer systems.

The term “MRI-compatible” means that a device is safe for use in an MRI environment and/or that a device that can operate as intended in an MRI environment and not introduce artifacts into MRI signal data. As such, if residing within the high-field strength region of the magnetic field of an MRI suite, the MRI-compatible device is typically made of a non-ferromagnetic MRI-compatible material(s) suitable to reside and/or operate in a high magnetic field environment. The term “RF safe” means that the lead or probe is configured to safely operate when exposed to RF signals, particularly RF signals associated with MRI systems, without inducing unplanned current that inadvertently unduly heats local tissue or interferes with the planned therapy or procedure. The term “shield” and derivatives thereof refer to an RF shield that can attenuate unwanted RF signal.

The term “high magnetic field” refers to magnetic fields above 0.5 T, typically between 1.5 T to 10 T, such as about 3.0 T and includes-high-field magnet closed bore and open bore MRI systems. MRI scanners are well known to those of skill in the art and include, for example, the SIGNA 1.5 T/3.0 T from GE Healthcare: the ACHEIVA 1.5 T/3.0 T and the INTEGRA 1.5 T from Philips Medical System; and the MAGNETOM Avanto, the MAGNETOM Espree, the MAGNETOM Symphony, and the MAGNETOM Trio, from Siemens Medical.

The term “fiducial marker” refers to a marker that can be electronically identified using image recognition and/or electronic interrogation, typically interrogation of CT or MRI image data. The fiducial marker can be provided in any suitable manner, such as, but not limited to, a geometric shape, a component on or in the device, optical or electrical tracking coils, a coating or fluid-filled component or feature (or combinations of different types of fiducial markers) that makes the fiducial marker(s) MRI-visible or CT visible with sufficient signal intensity (brightness) for identifying location and/or orientation information for the device and/or components thereof in space.

The term “grid” refers to a pattern of crossed lines or shapes used as a reference for locating points or small spaces, e.g., a series of rows and intersecting columns, such as horizontal rows and vertical columns (but orientations other than vertical and horizontal can also be used). The grid can include at least one fiducial marker. The grid can include associated visual indicia such as alphabetical markings (e.g., A-Z and the like) for rows and numbers for columns (e.g., 1-10) or the reverse. Other marking indicia may also be used. The grid can be provided as a flexible patch that can be releasably attached to the skin of a patient. For additional description of suitable grid devices, see co-assigned U.S. patent application Ser. No. 12/236,621 (U.S. Pat. No. 8,195,272), the contents of which are hereby incorporated by reference as if recited in full herein.

Embodiments of the present invention provide image-guided surgical systems 10 that can be configured to carry out or facilitate CT and/or MRI guided procedures, including, for example diagnostic and interventional procedures such as to guide and/or place interventional devices to any desired internal region of the body or object, including deep brain sites for neurosurgeries or other target intrabody locations for other procedures. The object can be any object and may be particularly suitable for animal and/or human subjects. For example, the system and/or devices thereof can be used for gene, e.g., antibody, and/or stem-cell based therapy delivery or other therapy delivery to intrabody targets in the brain, heart, lungs, liver, kidney, ovary, stomach, intestine, colon, spine or to other locations. In addition, embodiments of the systems can be used to treat cancer sites.

In some embodiments, the systems can be used to ablate tissue and/or deliver pharmacologic material in the brain, heart or other locations. In some embodiments, it is contemplated that the systems can be configured to treat AFIB, deliver stem cells or other cardio-rebuilding cells or products into cardiac tissue, such as a heart wall, via a minimally invasive MRI guided procedure while the heart is beating (i.e., not requiring a non-beating heart with the patient on a heart-lung machine).

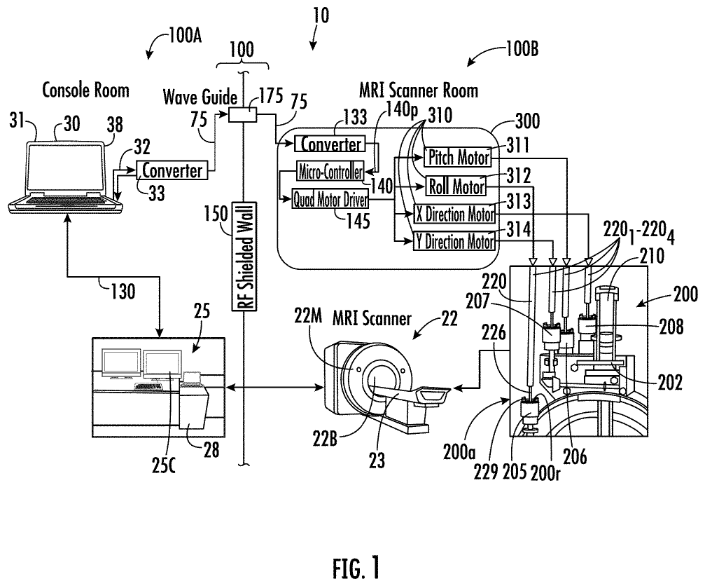

Turning now to the figures, FIG. 1 shows an exemplary MRI suite 100 with a control room 100A (also interchangeably referred to as a “console room”) and a separate MRI scanner room 100B that holds an MRI scanner 22 comprising a magnet 22M, typically a high field magnet. The magnet 22M can comprise a bore 22B that can have a closed bore or open bore configuration. The MRI scanner 22 can include a patient support table 23 that can retract into and out of the bore 22B of the magnet 22M as is well known to those of skill in the art.

A RF shielded wall 150 can reside between the control room 100A and the MRI scanner room 100B. A wave guide 175 (as discussed in the Background) can extend between the two rooms 100A, 100B through the RF shielded wall 150.

The image-guided surgical system 10 can include components in both the control room 100A and the MRI scanner room 100B. Certain components (e.g., motors 310 coupled to the actuators 200a) in the MRI scanner room 100B can be controlled by components (e.g., the computer system 31) in the control room 100A. Thus, the computer system 31 of a surgical procedure workstation 30 of the surgical navigation system 10 in the control room 100A can be in communication with a plurality of actuators 200a of a trajectory guide 200 in the MRI scanner room 100B.

Optionally, at least one fiber optic cable 75 can be routed through the wave guide 175 and couple the computer system 31 in the control room 100A to a housing 300 comprising a plurality of motors 310 in the MRI scanner room 100B. The computer system 31 can connect to a communications cable 32 which can couple to an input of a USB to fiber optic converter 33. The USB to fiber optic converter 33 can provide an output that is coupled to the fiber optic cable 75. The USB to fiber optic converter 33 can reside in the control room 100A.

It is contemplated that wireless communication systems, optionally BLUETOOTH, WiFi, LTE and/or infrared systems, may be used to couple the computer system 31 to the motors 310 in lieu of the at least one fiber optic cable 75 or with the at least one fiber optic cable 75.

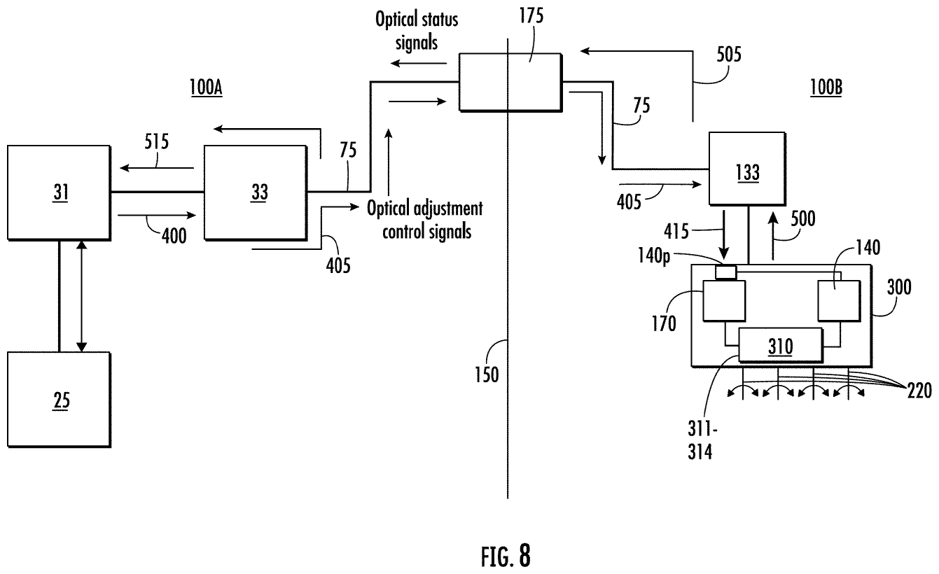

The surgical procedure workstation 30 of the surgical system 10 can comprise a display 38 that provides images and information to a user during a surgical procedure. The computer system 31 of the surgical procedure workstation 30 can be configured to provide a defined workflow(s) of actions including identifying a desired trajectory to a target surgical site using a targeting cannula Tc (FIG. 9) held by the trajectory guide 200. The computer system 31 can be configured to calculate positional adjustments of the actuators 200a for moving the trajectory guide 200 to provide the desired trajectory.

The computer system 31 can be coupled to a computer system 25 of the MRI scanner console 25C, also in the control room 100A, via a communications cable 130. The computer system 25 of the MRI scanner console 25C controls operation of the MRI scanner 22, e.g., applies selected and/or defined pulse sequences, slice selection, scan planes, signal acquisition (transmit excitation pulses and/or receive MR data of the patient) and the like. The computer system 25 in the control room 100A can communicate with or be at least partially installed in a control cabinet 28 with RF amplifiers, gradient amplifiers and/or circuits associated therewith.

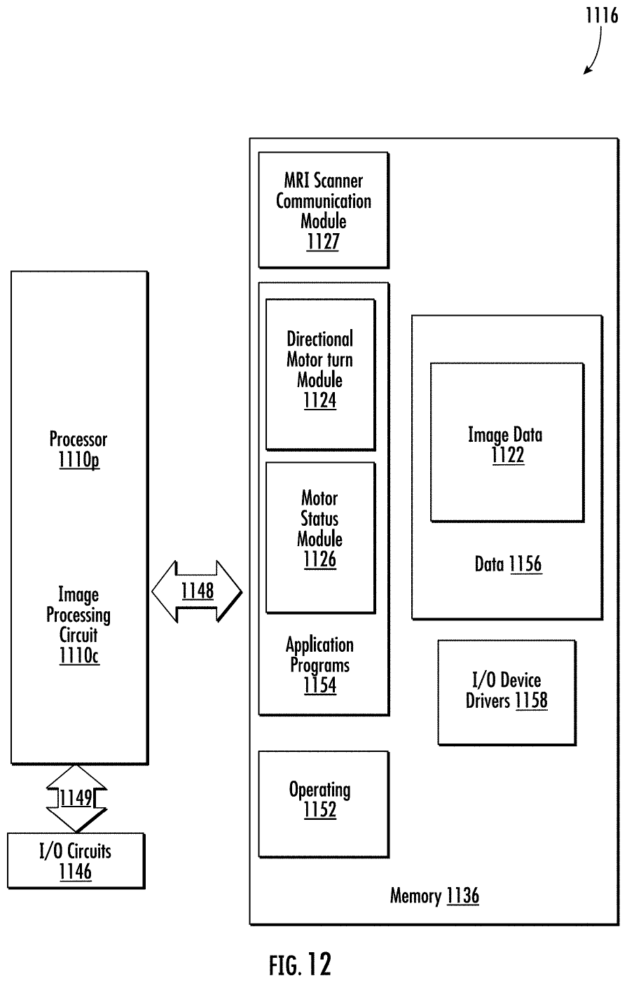

The image-guided surgical system 10 can include at least one housing 300 in the MRI scanner room 100B that holds a plurality of components that communicate with the computer system 25 in the control room 100A, optionally including motors 310 coupled to respective actuators 200a of the trajectory guide 200. In other embodiments, the motors 310 are held adjacent respective actuators 200a, spaced apart from and outside the at least one housing 300 with control cables 1225 connecting the components in the housing 300 and the respective motors 310 (see, FIG. 13A et seq.).

The at least one housing 300 can also hold a fiber optic to USB converter 133 and at least one microcontroller 140.

The at least one housing 300 can be a unit housing that holds the motors 310, the controller 140 and the converter 133 or that holds only the controller 140 and the converter 133.

The motors 310 can be coupled to the at least one microcontroller 140. The at least one microcontroller 140 is in communication with the computer system 31 of the surgical procedure workstation 30 in the control room 100A whereby the computer system 31 provides instructions to the at least one microcontroller 140, that then provides instructions to one or more of the motors 310 for controlled directional movement of the one or more motors 310 for accurate positional turns of one or more of the actuators 200a.

The surgical systems 10 can be configured to provide bi-directional communication between the microcontroller 140 in the MR scanner room 100B and the surgical procedure computer system 31 in the MR control room 100A using the computer system 31 and the microcontroller 140 and to provide status updates to the computer system 31 of when completion of directional movement of one or more motors 310 has occurred, with the motors 310 being provided directional commands (right, left) controlled by the computer system and microcontroller. When one or more of the motors 310 has completed its commanded directional movement, a status signal can be transmitted to the computer system 31 which can then trigger the MR computer system 25 to commence new image acquisitions to verify an adjusted trajectory of the trajectory guide 200. The status signals can facilitate a time-efficient trajectory alignment protocol.

Each motor 310 can be coupled to a respective actuator 200a of the trajectory guide 200, optionally via a corresponding cable 220 or via a relatively short motor shaft coupler 1220 provided by a motor drive assembly 1200 (FIG. 13A, for example).

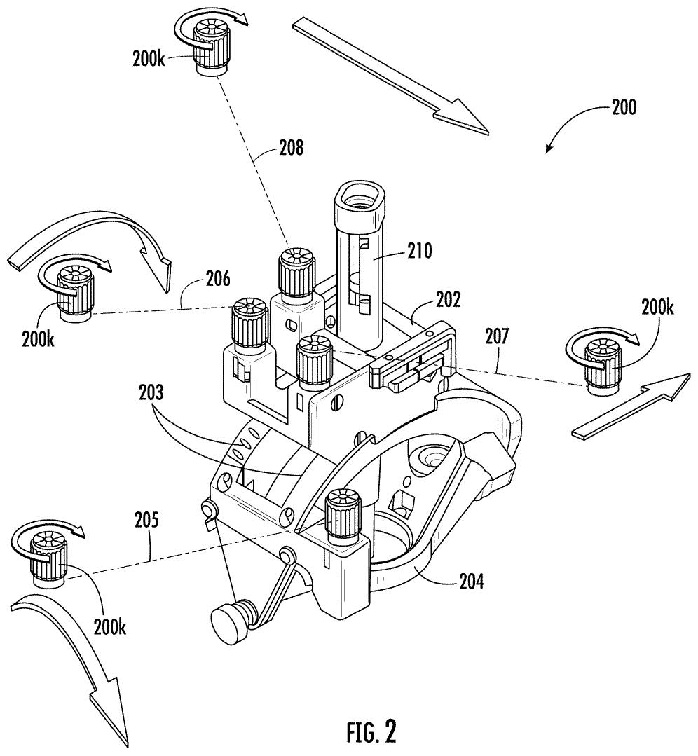

Referring to FIGS. 1 and 2, the plurality of actuators 200a can comprise a plurality of actuators 200a, typically between 2-4 actuators including a roll direction actuator 205, a pitch direction actuator 206 and optionally a Y direction actuator 207 and an X direction actuator 208. Where used, the X and Y-direction actuators 207, 208 can both be coupled to an X-Y table 202. The X-Y table 202 can be coupled to arcuate arms 203 projecting up from a base 204. The trajectory guide 200 can include a tower 210 defining a through-channel for releasably holding a targeting cannula Tc (FIG. 9) and/or an interventional device such as an ablation catheter, a fluid delivery or withdrawal cannula, a stimulation lead and the like.

Referring to FIG. 2, the roll and pitch actuators 205, 206, respectively, can comprise thumb wheels or knobs 200k and 1 revolution can correspond to a defined degree position adjustment, typically in a range of 1-5 degrees, such as 1 revolution equals about 4 degrees positional adjustment. The Y and X-direction actuators 207, 208, respectively, can also comprise thumb wheels or knobs 200k and one revolution can equate to a defined position change in a range of about 0.1 mm to about 5 mm, such as about 1 mm of directional change.

For additional discussion of suitable trajectory guides, see, U.S. application Ser. No. 12/134,412, and co-assigned U.S. patent application Ser. Nos. 12/236,950, and 14/515,105, the contents of which are hereby incorporated by reference as if recited in full herein.

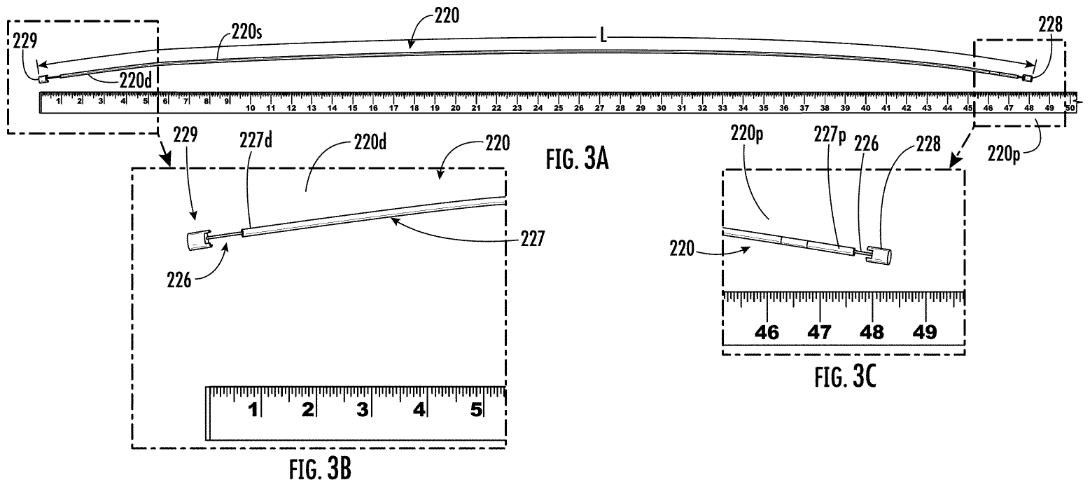

Referring to FIGS. 3A-3C, an example cable 220 is shown. Where used, the cable 220 can have longitudinally opposing proximal and distal ends 220p, 220d, respectively, with a length L. The length of the cables 220, (shown as cables 220₁-220₄ in FIG. 1), can have a common length L or a length L that is within about 1-5 inches of each other. The cables 220 can have a length L that is in a range of about 24 inches to about 100 inches, more typically in a range of about 36 inches to about 60 inches such as about 48 inches.

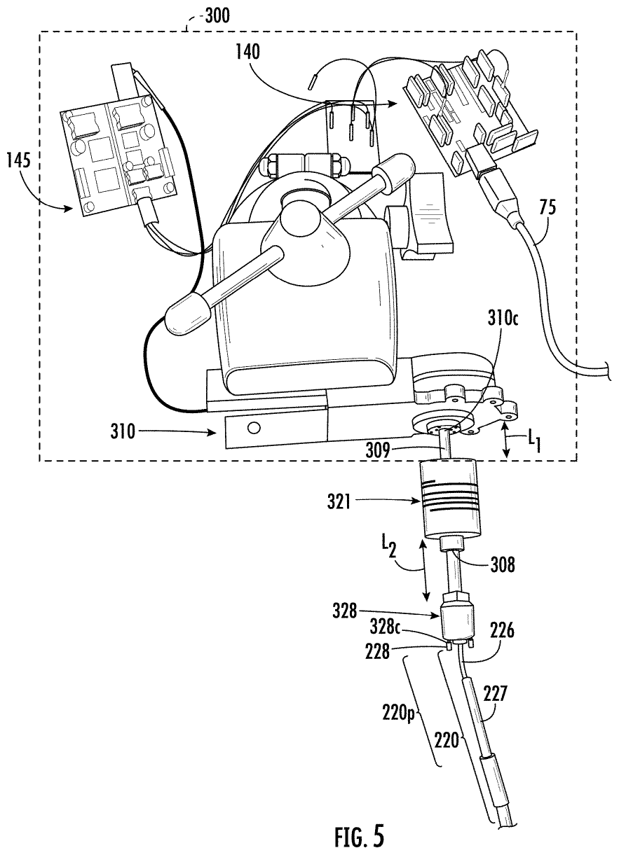

In some embodiments, the cables 220 can comprise torque transfer shafts 220s. The torque transfer shafts 220s can comprise a rigid or semi-rigid internal cable 226 and an outer shaft 227 enclosing the internal cable 226. The term “semi-rigid” means that the referred to object, e.g., in this case, the cable 220 has a self-supporting shape and but may flex side-to-side when assembled to the trajectory guide 200 and/or a corresponding motor 310, optionally via flex coupler 321 (FIG. 5).

In some particular embodiments, the internal cable 226, where used, can have a spiral rib(s) on an outer surface that can provide increased surface area to bond with an adhesive or filler material and fixedly couple to the outer shaft 227. The internal cable 226 can have a maximal outer diameter that is in a range of about 0.01 inches to about 0.3 inches. The outer shaft 227 can have a maximal outer diameter that is in a range of about 0.02 inches to about 0.5 inches.

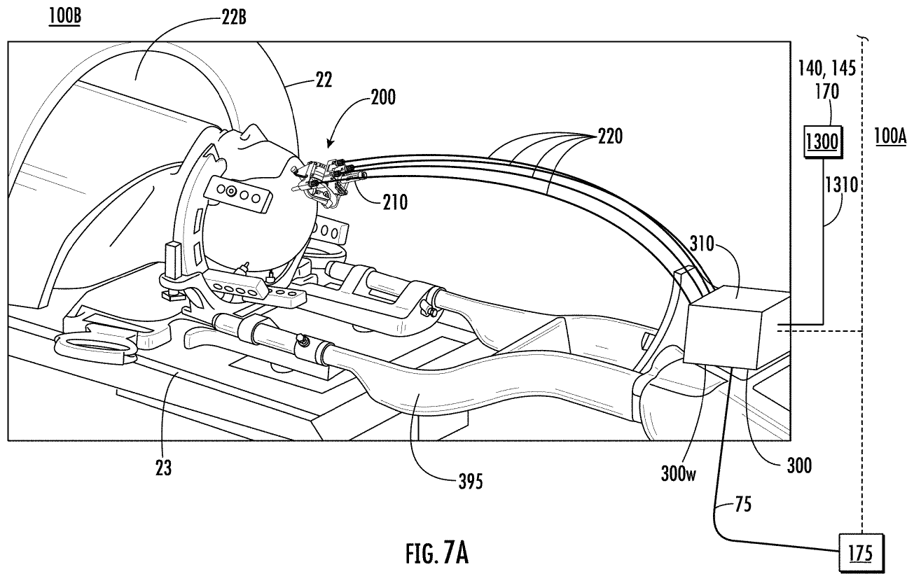

In some embodiments, in an assembled position, the shape of one or more of the cables 220 can be arcuate over at least a major portion of its length and extend in a curvilinear shape between opposing ends (FIGS. 3A, 7). This curvilinear shape can be pre-formed, self-supporting and retained based on a semi-rigid, malleable configuration of the cable(s) 220. as shown in FIG. 3A. The cable 220 can have sufficient rigidity to provide a defined, constant external inertia load for a respective motor 310, in use.

Where used, the internal cable 226 can comprise non-ferromagnetic material such as, for example, Nitinol steel. Where used, the outer shaft 227 can comprise a different non-ferromagnetic material than the internal cable 226. The internal cable 226 can have a solid core. The internal cable 226 can extend out of a distal end 227d of the outer shaft 227 and couple to a connector 229. The internal cable 226 can extend out of a proximal end 227p of the outer shaft 227 and couple to a connector 228. The extension lengths can be in a range of 0.1 inches to about 2 inches, in some embodiments. Thus, the internal cable 226 can have a continuous length that is longer than the length of the outer shaft 227. The outer shaft 227 can be fixedly coupled to the internal cable 226 via a non-ferromagnetic filler material to provide additional structural support over a length of the cable 220.

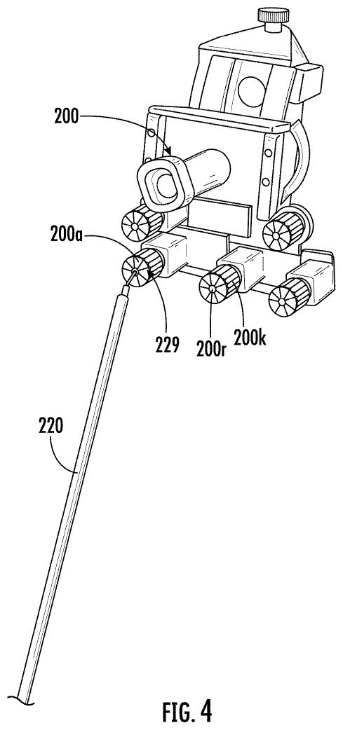

Referring to FIG. 4, the connector 229 at the distal end portion 220d of the cable 220 can be fixedly coupled to a respective actuator 200a of the trajectory guide 200 to rotate as a unit with the respective actuator 200a to provide controlled rotational movement of the actuator 200a in clockwise and counterclockwise directions. The connector 229 can be fixedly attached to a control thumbwheel or knob 200k of a respective actuator 200a. The connector 229 can be configured as a connector insert that is held by a slot, recess or channel 200r in an outer surface of the thumbwheel or knob 200k.

The connector 228 at the proximal end portion 220p of the cable 220 can be directly or indirectly coupled to a motor 310, such as a piezo motor 310p. The motor 310 can comprise or couple to a motor shaft 310s that couples to the cable 220.

Referring to FIG. 5, the cable 220 can optionally couple to a flexible shaft coupler 321. The flexible shaft coupler 321 can receive a first shaft 309 that has a first length “L₁” that extends out of one end of the coupler 321 and into the receiving channel 310c of the motor 310 and with a second shaft 308 that extends out of the other end of the coupler 321 with a second length “L₂” to a control knob connector 328.

Where used, the first shaft 309 can be rigid and non-ferromagnetic. The first length L₁ can be less than the second length L₂. The first length L₁ can be in a range of 1-4 inches. The second length L₂ can be in a range of 2-6 inches. The first shaft 309 can be metallic and non-ferromagnetic. Where used, the second shaft 308 can be rigid or semi-rigid and have a diameter that is greater than that of the first shaft 309. The second shaft 309 can comprise a polymer and/or fiberglass. The first and second shafts 309, 308 can each have an outer diameter that is greater than the cable 226 of the torque transfer shaft 220.

The control knob connector 328 can be configured to receive the connector 228 at the proximal end 220p of the cable 220, e.g., the first connector of the cable 220. The control knob connector 328 can have a channel 328c that receives the connector 228 and attaches thereto to be able to rotate as a unit. The connector 328 can be configured as a substantial duplicate of the thumbwheel or control knob 200k of a respective actuator 200a of the trajectory guide 200. That is, the control knob connector 328 can have substantially the same external body configuration, e.g., it can be formed of the same material and have the same outer diameter size and length as the corresponding knob 200k of the actuator 200a.

The first and second shafts 308, 309 and the coupler 321 can all be non-ferromagnetic.

The motors 310 can be non-ferromagnetic motors. The motors 310 can be piezo motors or pneumatic motors, for example. For further details of an example piezo motor, see, e.g., U.S. Pat. No. 9,136,778 to Discovery Technology International, Inc., the contents of which are hereby incorporated by reference as if recited in full herein. By way of example, a commercially available piezoelectric motor is the non-magnetic rotary piezo motor RBS Series from DTI, Inc., Sarasota, Fla. MRI compatible pneumatic stepper motors are described in U.S. Pat. No. 10,024,160 and PCT/NL2017/050552, the contents of which are hereby incorporated by reference as if recited in full herein.

Referring to FIGS. 1, 5 and 6, the at least one housing 300 can also hold at least one motor driver 145 (such as, for example, an amplifier unit) that is coupled to one or a plurality of different motors 310 and to the microcontroller 140. In an example embodiment, the plurality of motors 310 can be four non-ferromagnetic stepper motors, such as a pitch stepper motor 311, a roll stepper motor 312, an X-direction stepper motor 313, and a Y-direction stepper motor 314, each stepper motor provided as the motors 310.

The pitch stepper motor 311 can be coupled to the pitch direction actuator 206. The roll stepper motor 312 can be coupled to the roll direction actuator 205. The X-direction stepper motor 313 can be coupled to the X-direction actuator 208. The Y-direction stepper motor 314 can be coupled to the Y-direction actuator 207.

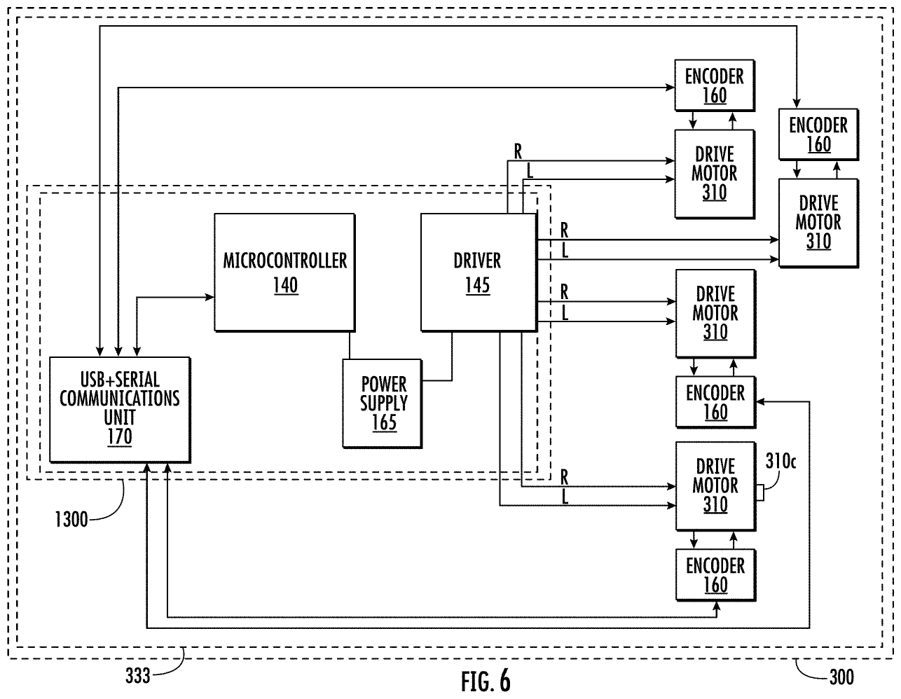

Each motor 310 can be coupled to a dedicated respective motor driver 145 and/or dedicated microcontroller 140 (four microcontrollers for four motors 310, for example, not shown). All the motors 310 can be coupled to a common, single microcontroller 140 and a single motor driver 145 (FIG. 6). Alternatively, two or three motors 310 can be coupled to a first microcontroller 140 and a first driver 145 while the others are coupled to another, second microcontroller 140 and/or second driver 145 (not shown).

Two or more motors 310 can serially or concurrently rotate a respective torque transfer shaft 220 to position the tower 210 of the trajectory guide 200 at an adjusted trajectory based on commands from the computer system 31 of the workstation 30. Referring to FIG. 6, the housing 300 can also enclose or include a power supply 165 and a USB and serial communications unit or module 170. Each motor 310 can be coupled to a respective encoder 160, such as an optical encoder.

The housing 300, 300′ can be EMI and/or RF-shielded 333 such as with copper fingers flashing or casings and/or epoxy at apertures and seams and can use appropriate externally accessible RF-attenuated connector(s) to reduce or eliminate any RF emissions from internal components.

In some embodiments, the power supply 165 and a USB and serial communications unit or module 170 and driver 145 are located in a separate electrically shielded enclosure 1300 (“drive control enclosure”) that can be placed in the scanner room 100B but further away from the bore of the MRI magnet relative to the other housing 300 with the motors 310 (“motor enclosure”). A suitable multiconductor electrical cable carries signals from the at least one motor driver 145 to the motors 310 and the output of the encoder back to the at least one motor driver 145. This approach can reduce or minimize electrical interaction between the MR scanner 22 and the motor driver 145, microcontroller 140, power supply 165 and USB serial communication unit 170 electronics. Electrical interaction can create artifacts in the MRI images. During the process of creating an MRI image very strong electromagnetic pulses are generated in the MRI scanner bore by the RF coil and gradient coils. These pulses can potentially cause the at least one motor driver 145, microcontroller 140, power supply 165 and USB serial communication unit 170 electronics to malfunction if not properly positioned and/or electrically isolated.

Referring to FIG. 7A, the motor enclosure/housing 300 can be coupled to the scanner bed 23. The motor enclosure/housing 300 can be supported by an attachment assembly 395 that extends longitudinally outward from the scanner bed 23. The motor enclosure/housing 300 can have a polygonal shape with at least one wall, shown as four sidewalls, connecting a ceiling and floor, as shown. Each shaft 309 and/or 308 (FIG. 5) can extend out of a single (side) wall 300w of the housing 300. In other embodiments, one or more shafts 309 and/or 308 connected to a respective motor channel 310c (FIG. 5) can extend out of a ceiling or end wall facing the trajectory guide 200. The at motor enclosure/300 can be coupled to the scanner bed 23 to translate in and out of the bore 22B of the magnet 22M while attached to the trajectory guide 200 so that there is no compression or extension of the cables 220 due to this motion.

In some embodiments, as shown in FIG. 7A, the motors 310 and cables 220 can travel in and out of the bore of the magnet while mounted/coupled to the scanner bed 23. A flexible multi-conductor cable assembly 1310, provided as a suitably shielded electrical cable, can carry electrical signals to the motor(s) 310 and encoder signals back to the driver electronics enclosure 1300, with the driver 145, microcontroller 140 and USB/serial communications module 170, also in the MRI scanner room 100B, that can be placed further away from the magnet, such as on the floor or a countertop or cabinet in a lower Gaussian field. The enclosure 1300 can be placed in a lower Gaussian field such as outside a 5 Gauss line in the scanner room 100B.

The motor enclosure/housing 300, the at least one fiber optic cable 310 and all internal components of the housing 300, including the motors 311-314, cables 220, connectors 228, 229, and flexible coupler 321 are made of non-ferromagnetic (MRI compatible) material and do not experience any significant magnetic force pull from the MRI superconducting magnet 22M. The housing 1300 and/or the motor driver 145, microcontroller 140 and power supply 165 may include minimally magnetic components.

Communication between the computer system 31 and the at least one motor driver 145 can be two-way. The at least one motor driver 145 can signal the computer system 31 once one or more of the motors 310 has finished turning the required angle.

Embodiments of the invention can provide the communication between the computer system 31 and the microcontroller 140 and the at least one motor driver 145 using one or a plurality of fiber optic cables 75

FIG. 7B illustrates an example motor enclosure/housing 300 with the motors 311-314. The motor enclosure 300 can include a plurality of connectors 319, for example, SMT connectors, that couple to respective drive motors 310 and to the cable assembly 1310 via a connector 303 attached to the enclosure 300. The drive control enclosure 1300 can include a connector 1303 that couples the motor drivers 145 to the cable assembly 1310. At least one converter 133 (fiber optic to USB) can also be held in the enclosure 1300. The drive control enclosure 1300 and the motor enclosure 300 can be non-ferromagnetic. The encoders 160 are typically held in the motor enclosure 300. The cable assembly 1310 can have a length of 5-10 meters, in some embodiments. The fiber optic cable 75 can couple to the drive control enclosure 1300. Only optical control signals from computer(s) (25,31) outside scanner room, optionally in the MRI control room 100A, pass through waveguide 175 (FIG. 1). Further, the cable assembly 1310 may include electrical conductors that may allow external RF to leak into the scanner room, which if placed in certain locations may create artifacts in the MR images. However, the cable assembly 1310 can be made sufficiently long to place the drive control enclosure 1300 outside the 5 Gauss line. This can be important as some of the components on the motor driver 145, e.g., on the driver board 145b, power supply 165, and micro controller 140 may be ferromagnetic and/or otherwise need to be positioned away from the MRI magnet.

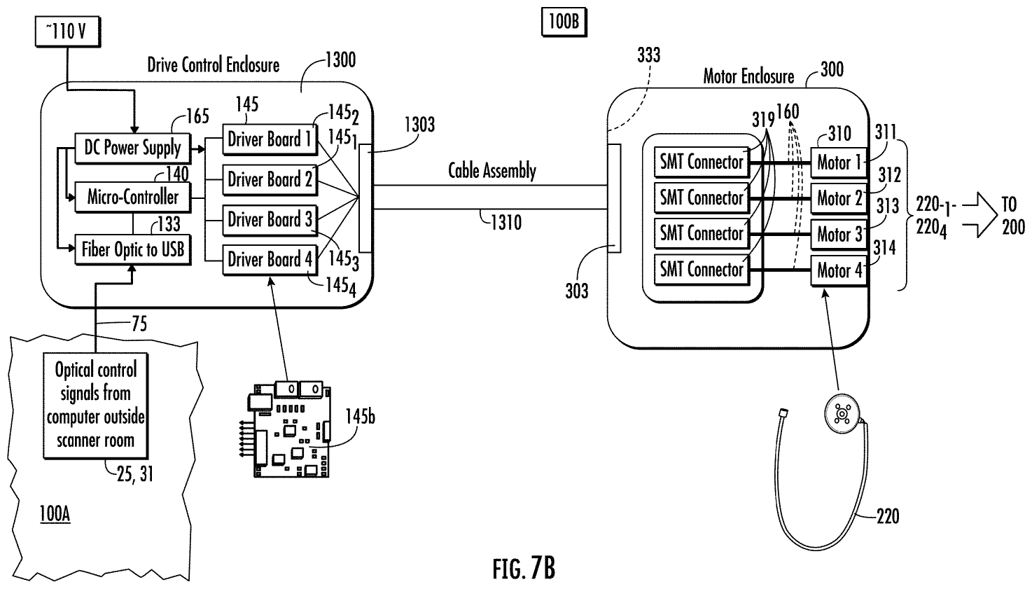

Referring to FIG. 8, as shown, the computer system 31 of the surgical procedure workstation 30 and/or surgical procedure circuit thereof can be configured to communicate with the MRI scanner computer system 25 and can be configured to control the motors 310 using adjustment signals 400, converted at converter 33 to optical signals 405 in the control room 100A, then down converted at converter 133 to electrical signals 415 in the scanner room 100B to automatically adjust (rotate or turn) the actuators 200a to position the trajectory guide 200 in the desired trajectory. The controller 140 can send status information via signals 500, converted at converter 133 to optical signals 505, then converted at converter 33 in the control room 100A to electrical signals 515 for the USB of the computer system 31 of the surgical procedure workstation 30.

Referring to FIGS. 1 and 8, the computer system 31 can be configured to provide the surgical trajectory adjustment calculations and directional turn instructions in the MRI console room 100A. The computer system 31 can be configured to communicate adjustment values to the controller 140 in the MRI scanner room 100B via the at least one fiber optic cable 310. Thus, the surgical system 10 can be configured so that the workstation 30 and/or computer system 31 thereof provides instructions to the controller 140, (optionally a microcontroller or digital signal processor).

Referring to FIG. 8, the communication signals 400 from the computer system 31 are transmitted to converter 33 then provided as optical signals 405 through the one or more fiber optic cable 75 to be down converted via converter 133 inside the scanner room 100B and fed as serial communication signals 415 to the USB port 140p of the controller 140.

The at least one fiber optic cable 75 is electrically nonconductive and prevents or inhibits outside radio frequency signals from penetrating through the shield (Faraday Cage) of the MRI scanner room 100B. The MRI scanner 22 is enclosed in the shielded room/Faraday cage as external radio frequency signals will interfere with the functioning of the ultra-sensitive radio frequency receivers of the MRI scanner. This interference appears as artifacts in the MRI images.

The controller 140 can be configured to programmatically decode the motor turn instructions, send appropriate signals to (start and stop inputs) the motors 310, optionally by sending commands to the associated driver 145 or to a quad motor driver 145 (FIGS. 1, 6)

The controller 140 can also be configured to provide status updates to the surgical procedure workstation 30 and/or computer system 31 thereof. The status updates can include one or more of: one or more driver still in motion for adjusting an actuator 200a, motor completed its commanded turn motion to turn a respective actuator for the current positional turn direction instructions.

In some embodiments, after executing all commands to turn the actuators 200a, the microprocessor 140 sends back a defined code (which indicates the completion of all commands), as well as the value of the number of counted pulses (4 bytes), when the last command is executed. A rotary encoder can have a resolution in a range of about 100-300 μrad, such as about 196 μrad (32,000 PPR) after interpolation and quadrature detection. Thus, for example, 32,000 pulses can correspond to one complete turn of the motor shaft 310s.

Referring to FIGS. 6 and 8, the status updates can be provided based on signals from encoders 160 of motors 310. The encoders 160 can be coupled to a USB and Serial Communications unit 170 in the housing 300 and the unit 170 can be coupled to the controller 140 and/or directly provide the status updates signals 500 to the converter 133, then corresponding optical signals 505 can be transmitted via the at least one fiber optic cable 75 to the converter 33, then as communication signals 510 to the computer system 31.

After each positional adjustment or a set of adjustments of one or more actuators 200a by one or more motor 310, the workstation 30 and/or computer system 31 thereof can be configured to direct and/or trigger the computer system 25 of the MRI scanner console to acquire new sets of images.

Referring to FIG. 9, from these new images, the current position and orientation of the targeting cannula Tc (FIG. 9) is identified through segmentation of images from orthogonal scan planes 1500₁, 1500₂, whereby slices can be aligned and a trajectory provided by the trajectory guide 200 can be re-calculated and new actuator (e.g., thumb wheel) 200a adjustment values to move the trajectory guide to provide the desired trajectory are generated by the surgical workstation 30 and/or computer system thereof 31. The adjustment process can be repeated until a projected error is below a defined acceptable value either set as a default or set as a defined value by a respective user or until the actuator(s) 200a (FIG. 1) are maxed out in a particular direction.

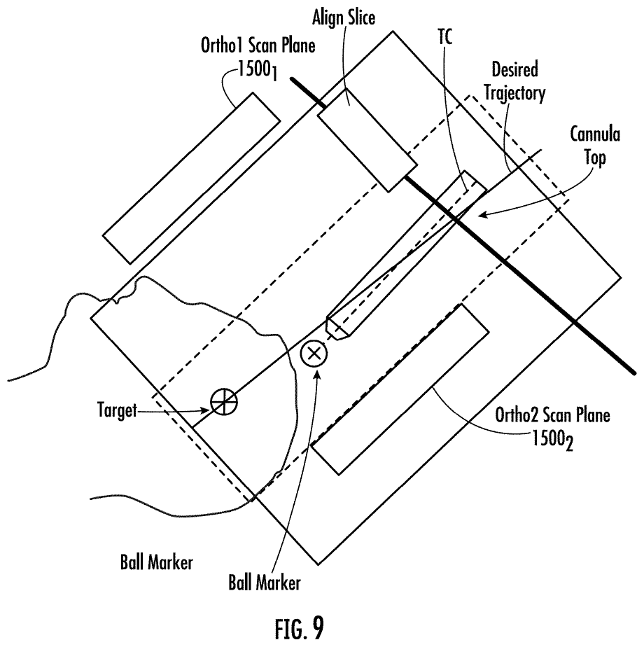

Referring to FIG. 10, in some embodiments, the computer system 25 of the MRI scanner can be configured with server software that can be activated on the MR scanner host computer system 25 by a user. Commercially available Scanners with this configuration include Siemens—Access-I, GE—EXSI, Phillips—XTC. A physical connection can be established between the surgical navigation computer system 31 and the scanner host computer system 25 via a dedicated local area network (LAN) router. The surgical computer system 31 can provide software that runs as a remote client and establishes a secure bi-directional communication with the MRI scanner host computer system 25. This can be configured as a client-server relationship between the two computer systems 25, 31. As is well known to those of skill in the art, MR scanner host computer server software contains several dedicated remote service interfaces that allow a remote client like the surgical procedure workstation 30 to control parameters and execute image exams and/or image acquisitions on the MRI scanner 22.

In an example set-up, as shown in FIG. 10, the surgical navigation computer system 31 can be configured with software that comprises software technology based on ‘Representational State Transfer’ (RST) and ‘Java Script Object Notation’ (JSON) to communicate with the server running on the computer system 25 of the MRI scanner (e.g., MR Scanner host system).

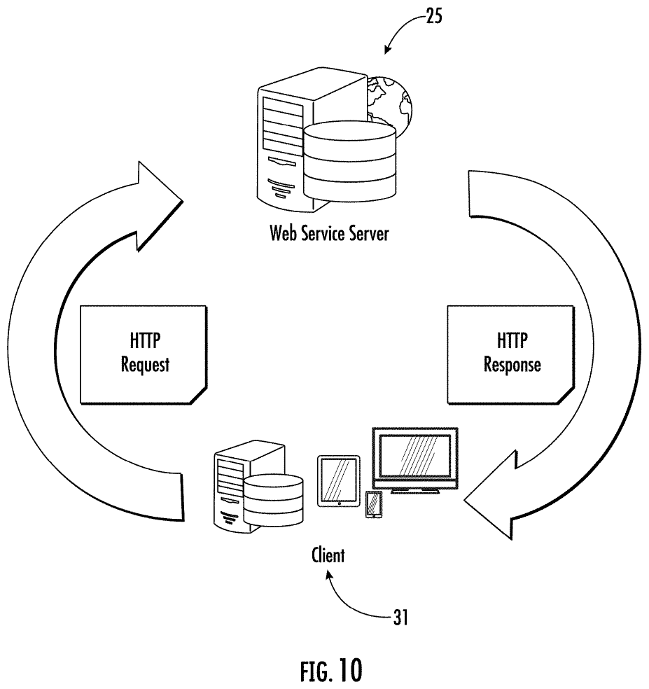

For example, in block 610 of FIG. 11, the workstation 30 can issue commands via ‘HyperText Transfer Protocol’ (HTTP) requests to transfer image plane parameters to the computer system 25 of the MRI scanner. In return, it receives confirmation via HTTP from the computer system 25 of the MRI scanner that the scan plane parameters have been received and the scanner is ready to execute imaging.

The surgical procedure system 10 can define a set of actions (a workflow) for the image-guided surgical navigation system 10. See, for example, U.S. Pat. No. 8,315,689B2, entitled MRI Surgical Systems for Real-Time Visualizations Using MRI Image Data And Predefined Data Of Surgical Tools (which describes components of, inter alia, an image-guided system also known as the CLEARPOINT® image-guided surgical system from MRI Interventions, Inc., Irvine, Calif.), the contents of which are hereby incorporated by reference as if recited in full herein.

During an interventional surgical procedure, the trajectory guide 200 is adjusted to a desired trajectory based on directions from the computer system 31 which iteratively (1) acquires MRI images at certain imaging planes in 3D space, (2) uses the acquired images to locate a position in three dimensional image space of a Tc (FIG. 9) position in 3D space, (3) automatically calculates a rotation direction and number of turns to be applied to specific actuators 200a via thumb wheels or knobs 200k located on the trajectory guide 200 and then (4) automatically drives the thumbwheels using remote control inputs and motors coupled to the torque transfer shafts 220 to make required adjustments. This entire process is repeated until the user is satisfied with the projected error indicated by the software or based on a default or selected threshold or maximal acceptable error.

In embodiments of the present invention, the surgical procedure workflow can occur after the trajectory guide is mounted to or adjacent the patient head and the trajectory guide adjustment can be broken into two primary adjustment actions. That is, the trajectory guide 200 can be directly attached to a skull of a patient or held by a support assembly such as that described in U.S. patent applications number Ser. Nos. 14/619,847; 62/964,340 (Attorney Dkt. 9450-137PR) and 62/968,210 (Attorney Dkt. 9450-138PR), the contents of which are hereby incorporated by reference as if recited in full herein.

A rough or preliminary adjustment can be provided by an ‘Align’ action(s) in the surgical procedure workflow. This can be followed by a finer adjustment or an ‘Adjust’ action(s) in the surgical procedure workflow. The trajectory guide 200 can couple to a targeting cannula (Tc, FIG. 9) which comprises an MRI visible fluid. See, U.S. Pat. No. 9,042,958, the content of which is hereby incorporated by reference as if recited in full herein. Images of this cannula Tc (FIG. 9) are used by the surgical procedure computer system 31 to determine its location within 3D space inside the MRI magnet. After segmenting the cannula, the surgical procedure computer system 31 (e.g., software algorithms, circuits or modules) can then calculate one or more actuator 200a adjustment, e.g., the thumbwheel or knob 200k turn adjustment needed for one or more of the pitch, roll, X or Y actuators 205-208 to align the trajectory guide 200 to the desired trajectory. FIG. 11 provides a summary of example trajectory guide adjustment steps or actions.

FIG. 11 is a flow chart of exemplary operations that can carry out alignment adjustments according to embodiments of the present invention. Motors can be physically coupled to the trajectory guide (using elongate torque transfer shafts) (block 600). The surgical procedure workstation can connect to and control the MRI scanner to direct the MRI scanner to perform imaging and carry out motorized adjustment of the trajectory guide (block 605). Transfer slice plane parameters are sent to the MRI console computer (block 610). A single slice image acquisition is initiated through the top of the (targeting) cannula (block 615). The acquired images of the single slice are downloaded (block 620). The projected error is calculated to determine if it is less than a desired value (typical 2D projected error is <2 mm) (block 630). If the projected error is not less than the desired value (i.e., 2 mm), the pitch and roll motors are turned via commands from a fiber optic interface (block 625). Then the steps of the operation are repeated starting with the step of initiating a single slice image through the top of the cannula (block 615). Typically, three-four (3-4) iterations are needed to arrive at the “gross” or first adjusted position.

If the projected error is less than the desired value (i.e., 2 mm), the orthogonal #1 and #2 image stack plane parameters are set on and/or to the MRI console (block 635). Next, acquisition of a first stack of contiguous orthogonal slices that cover the entire targeting cannula is initiated (block 640). The acquired images of the first stack of contiguous orthogonal slices are downloaded (block 645). Acquisition of a second stack of contiguous orthogonal slices that cover the entire targeting cannula is initiated (block 650). The acquired images of the second stack of contiguous orthogonal slices are downloaded (block 655). The projected error is again calculated to determine if the projected error is less than a desired value (typical 2D projected error is <1 mm) (block 665). If the projected error is not less than the desired value (i.e., about 1 mm or less), one or more of the pitch and roll motors and/or X and Y stage motors (where used) can be directionally turned via fiber optic interface (block 660). The steps of the operation are repeated starting with downloading the acquired images of the first stack of contiguous orthogonal slices (block 645). Typically, two-four (2-4) iterations are needed to achieve a sufficiently accurate defined intrabody trajectory. If the projected error is less than the desired value (i.e., about 1 mm or less), the trajectory alignment goal is met and the position adjustment can be ended.

As discussed above, MRI compatible stepper motors 310 can be used to make the required positional adjustments. The automated system can reduce the time taken to finalize the trajectory frame adjustment from 60 minutes to less than about 10 minutes. This savings in time can provide a procedure value to surgeons and facilitate an ease in performing the surgery with accuracy and overall reduction in cost.

The controller 140 can have embedded software that decodes motor turn instructions, sends appropriate signals to start and stop motors 310 by sending commands to the individual motor drivers 145 or to a quad motor driver that communicates with each motor 310. The controller 140 can also provide status updates to the surgical procedure workstation 30. After each adjustment, the computer system 31 of the surgical procedure workstation 30 can trigger the computer system 25 of the MRI scanner to acquire new sets of images. From these new images, the position of the targeting cannula Tc is segmented, a trajectory is re-calculated and new thumb wheel/knob adjustment values are generated by the surgical procedure workstation 30. The adjustment process can be repeated until projected error is below a certain defined value that can be a default value or a predetermined by the user or the adjustment knobs are maxed out.

The system 10 can be configured to render or generate real time visualizations of the target anatomical space using MRI image data and predefined data of the trajectory guide and at least one surgical tool to segment the image data and place the trajectory guide and/or tool in the rendered visualization in the correct orientation and position in 3D space. Embodiments of the present invention may include steps, features, aspects, components, procedures and/or systems as disclosed in U.S. patent application Ser. No. 12/236,854, published as U.S. Published Patent Application No. 2009/0171184, the disclosure of which is incorporated herein by reference. Embodiments of the present invention use the surgical support system 10 with an automated or semi-automated surgical procedure system comprising defined workflows and DICOM communication with the computer system 25 of the MR Scanner. See, e.g., U.S. Pat. No. 10,105,485, the contents of which are hereby incorporated by reference as if recited in full herein.

According to some embodiments, the systems are configured to provide a substantially automated or semi-automated and relatively easy-to-use MRI-guided system with defined workflow steps and interactive visualizations. In particular embodiments, the systems define and present workflow with discrete steps for guiding the alignment of the targeting cannula Tc held by the trajectory guide 200 to a planned trajectory, monitoring the insertion of a delivery cannula, and adjusting the (X-Y) position in cases where the placement needs to be corrected.

A relatively long multiple (e.g., four or more) conductor, fiberoptic or optical cables 75 can be used. The fiber optic cable 75 can be between about 5-50 meters long or longer, such as about 5 meters, about 6 meters, about 7 meters, about 8 meters, about 9 meters, and about 10 meters, about 20 meters, about 30 meters, about 40 meters, and about 50 meters, in some embodiments. The cable 75 can be obtained from any suitable supplier such as, for example, Lastar Cables 2 Go, Irvine, Calif. and Opticis, Sungnam City, Rep. of Korea. The interface at the housing 300 can include an EMI filter, RF shielding and a copper contact strip 333 from any suitable supplier such as Laird or Shaffner EMC referenced above.

As discussed above, other communications systems may be used that do not require fiber optic cables, such as BLUETOOTH and/or infrared systems.

Embodiments of the present invention can be configured to carry out diagnostic and interventional procedures such as to guide and/or place interventional devices to any desired internal region of the body or object but may be particularly suitable for neurosurgeries for ablation or fluid delivery or withdrawal. The object can be any object and may be particularly suitable for animal and/or human subjects. The system 10 can be used for gene and/or stem-cell based therapy delivery or other neural therapy delivery and allow user-defined custom targets in the brain or to other locations. In addition, embodiments of the systems can be used to ablate tissue in the brain or other locations. In some embodiments, it is contemplated that the systems can be configured to treat AFIB in cardiac tissue, and/or to deliver stem cells or other cardio-rebuilding cells or products into cardiac tissue, such as a heart wall, via a minimally invasive MRI guided procedure while the heart is beating (i.e., not requiring a non-beating heart with the patient on a heart-lung machine).

Examples of known treatments and/or target body regions are described in U.S. Pat. Nos. 6,708,064; 6,438,423; 6,356,786; 6,526,318; 6,405,079; 6,167,311; 6,539,263; 6,609,030 and 6,050,992, the contents of which are hereby incorporated by reference as if recited in full herein.

Embodiments of the present invention may take the form of an entirely software embodiment or an embodiment combining software and hardware aspects, all generally referred to herein as a “circuit” or “module.” In some embodiments, the circuits include both software and hardware and the software is configured to work with specific hardware with known physical attributes and/or configurations. Furthermore, the present invention may take the form of a computer program product on a computer-usable storage medium having computer-usable program code embodied in the medium. Any suitable computer readable medium may be utilized including hard disks, CD-ROMs, optical storage devices, a transmission media such as those supporting the Internet or an intranet, or other storage devices.

Computer program code for carrying out operations of the present invention may be written in an object-oriented programming language such as Java®, Smalltalk or C++. However, the computer program code for carrying out operations of the present invention may also be written in conventional procedural programming languages, such as the “C” programming language. The program code may execute entirely on the user's computer, partly on the user's computer, as a stand-alone software package, partly on the user's computer and partly on another computer, local and/or remote or entirely on the other local or remote computer. In the latter scenario, the other local or remote computer may be connected to the user's computer through a local area network (LAN) or a wide area network (WAN), or the connection may be made to an external computer (for example, through the Internet using an Internet Service Provider).

The present invention is described in part below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams of certain of the figures herein illustrate exemplary architecture, functionality, and operation of possible implementations of embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order or two or more blocks may be combined, depending upon the functionality involved.

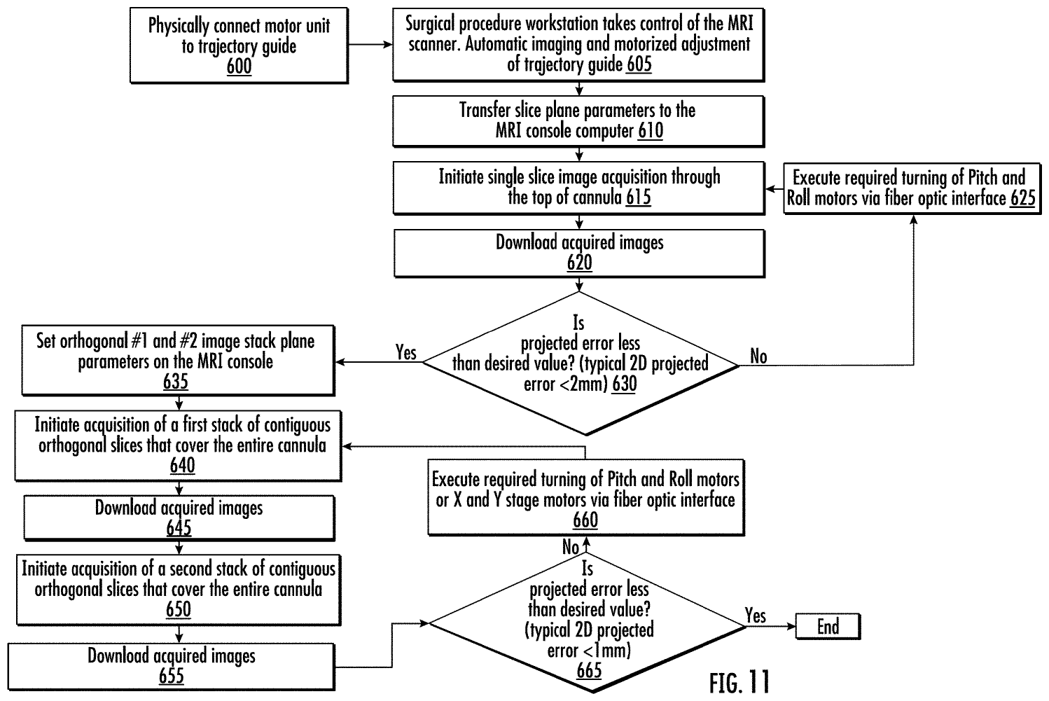

As illustrated in FIG. 12, embodiments of the invention may be configured as a data processing system 1116, which can include a (one or more) processors 1110p, a memory 1136 and input/output circuits 1146. The one or more processors 1110p can be part of the image processing circuit 1110c. The data processing system may be incorporated in, for example, one or more of a personal computer, database, workstation W, server, router or the like. The system 1116 can reside on one machine or be distributed over a plurality of machines. The processor 1110p communicates with the memory 1136 via an address/data bus 1148 and communicates with the input/output circuits 1146 via an address/data bus 1149. The input/output circuits 1146 can be used to transfer information between the memory (memory and/or storage media) 1136 and another computer system or a network using, for example, an Internet protocol (IP) connection. These components may be conventional components such as those used in many conventional data processing systems, which may be configured to operate as described herein.

In particular, the processor 1110p can be commercially available or custom microprocessor, microcontroller, digital signal processor or the like. The memory 1136 may include any memory devices and/or storage media containing the software and data used to implement the functionality circuits or modules used in accordance with embodiments of the present invention. The memory 1136 can include, but is not limited to, the following types of devices: ROM, PROM, EPROM, EEPROM, flash memory, SRAM, DRAM and magnetic disk. In some embodiments of the present invention, the memory 1136 may be a content addressable memory (CAM).

As further illustrated in FIG. 12, the memory (and/or storage media) 1136 may include several categories of software and data used in the data processing system: an operating system 1152; application programs 1154; input/output device drivers 1158; and data 1156. As will be appreciated by those of skill in the art, the operating system 1152 may be any operating system suitable for use with a data processing system, such as LABVIEW®, IBM®, OS/2®, AIX® or zOS® operating systems or Microsoft® Windows®95, Windows98, Windows2000 or WindowsXP operating systems, Unix or Linux™, IBM, OS/2, AIX and zOS are trademarks of International Business Machines Corporation in the United States, other countries, or both while Linux is a trademark of Linus Torvalds in the United States, other countries, or both and LABVIEW is a registered trademark of National Instruments Corporation. Microsoft and Windows are trademarks of Microsoft Corporation in the United States, other countries, or both.

The input/output device drivers 1158 typically include software routines accessed through the operating system 1152 by the application programs 1154 to communicate with devices such as the input/output circuits 1146 and certain memory 1136 components. The application programs 1154 are illustrative of the programs that implement the various features of the circuits and modules according to some embodiments of the present invention. Finally, the data 1156 represents the static and dynamic data used by the application programs 1154 the operating system 1152 the input/output device drivers 1158 and other software programs that may reside in the memory 1136.

The data 1156 may include MRI image data sets with metadata correlated to respective patients. As further illustrated in FIG. 12, according to some embodiments of the present invention, the application programs 1154 include a trajectory guide motor turn directional instruction module 1124. The application programs can also include a motor status module 1126 and an MRI scanner communication module 1127. The application programs 1154 may be located in a local server (or processor), local client, and/or database or a remote server (or processor), remote client and/or database, or combinations of local and remote databases and/or servers and/or clients.

While the present invention is illustrated with reference to the application programs 1154, and modules 1124, 1126 and 1127 in FIG. 12, as will be appreciated by those of skill in the art, other configurations fall within the scope of the present invention. For example, rather than being application programs 1154 these circuits and modules may also be incorporated into the operating system 1152 or other such logical division of the data processing system. Furthermore, while the application programs 1124, 1126 and 1127 are illustrated in a single data processing system, as will be appreciated by those of skill in the art, such functionality may be distributed across one or more data processing systems in, for example, the type of client/server arrangement described above. Thus, the present invention should not be construed as limited to the configurations illustrated in FIG. 12 but may be provided by other arrangements and/or divisions of functions between data processing systems. For example, although FIG. 12 is illustrated as having various circuits and modules, one or more of these circuits or modules may be combined or separated without departing from the scope of the present invention.

The computer system 25 of the MRI scanner can include a console that has a “launch” application or portal for allowing communication to the circuit 31 of the surgical procedure workstation 30.

The scanner console can acquire volumetric T1-weighted (post-contrast scan) data or other image data (e.g., high resolution image data for a specific volume) of a patient's head or other anatomy. In some embodiments, the console can push DICOM images or other suitable image data to the workstation 30 and/or computer system 31. The workstation 30 and/or computer system 31 can be configured to passively wait for data to be sent from the computer system 25 of the MR scanner 22 and the workstation 30 does not query the Scanner or initiate a communication to the Scanner. In other embodiments, a dynamic or active communication protocol between the circuit computer system 31/workstation 30 and the computer system 25 of the Scanner may be used to acquire image data and initiate or request particular scans and/or scan volumes.

Also, in some embodiments, pre-DICOM, but reconstructed image data, can be sent to the circuit workstation 30 for processing or display. In other embodiments, pre-reconstruction image data (e.g., substantially “raw” image data) can be sent to the workstation 30 for Fourier Transform and reconstruction. Embodiments of the present invention use the surgical support system 10 with an automated or semi-automated surgical navigation system comprising defined workflows and DICOM communication with an MR Scanner. See, e.g., U.S. Pat. No. 10,105,485, the contents of which are hereby incorporated by reference as if recited in full herein.

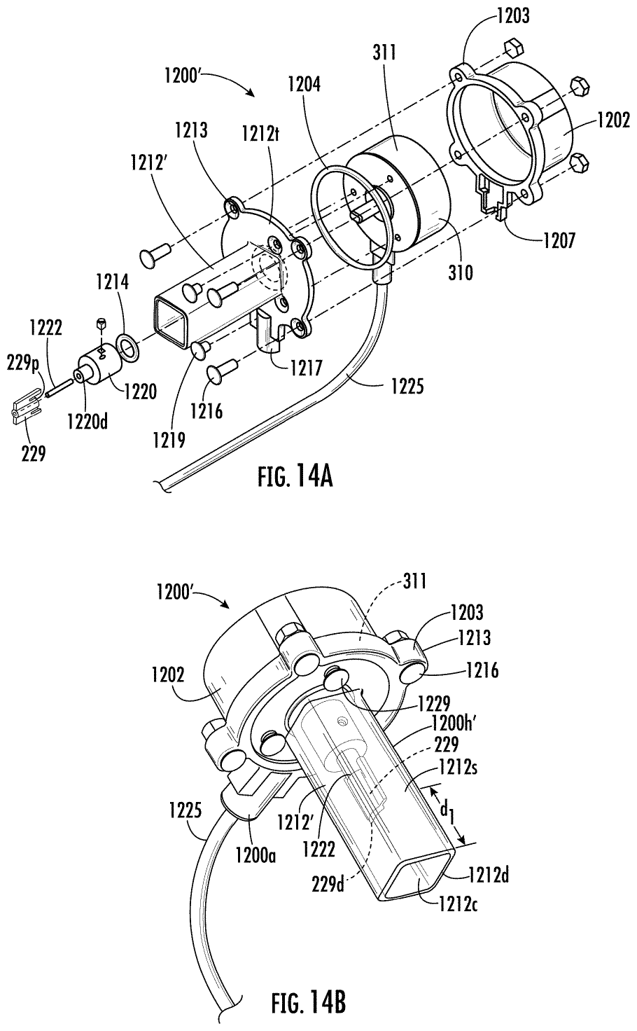

Turning now to FIGS. 13A and 13B, another embodiment of a drive system comprising a motor assembly 1200 that provides a respective drive motor 310 for one of the actuators 200a (FIG. 15A) is shown. In this embodiment, the motor assembly 1200 comprises a motor assembly housing 1200h that can sealably enclose a respective (single) motor 310. The sealed configuration allows the motor assembly housing 1200h and components thereof to be sterilized (EtO or steam) as an assembled unit to provide a medical grade sterile motor assembly 1200 for surgical purposes. To be clear, the term “sterile” refers to regulatory guidelines for surgical devices, such as U.S. Food and Drug Administration (FDA) guidelines. Thus, the motor assembly 1200 can reside adjacent a patient during a medical procedure and can be configured to be in a medical grade sterile state.

The motor assembly 1200 can be light weight and size to avoid introducing torque forces onto the trajectory guide 200. The motor assembly 1200, absent the control cable 1225, can have a weight that is in a range of about 50 grams to about 100 grams. The housing 1200h can have a compact maximal height H or length dimension “L” and maximal width “W” dimension, with each in a range of about 1.5 inches to about 3.5 inches, such as each within a range of about 1.5 inches, about 1.75 inches about 2 inches, about 2.5 inches, about 3 inches and about 3.5 inches. The H, L and W dimensions can be different. The L dimension can be greater than the W dimension. The housing 1200h can be non-ferromagnetic and may optionally be molded from a PEEK or polycarbonate material. The housing 1200h can have at least a portion that is visually transmissive at least adjacent the drive wing receiving region to facilitate ease of installation. The housing 1200h or a lower portion thereof can be provided as a translucent and/or transparent non-ferromagnetic material such as a transparent or translucent polymer

The housing 1200h can have a chamber 1200c that receives the motor 310. The chamber 1200c can be provided by an enclosure cap 1202c. The motor assembly 1200 can include a seal 1204, such as an O-ring, that is held in a circumferential recess of the enclosure cap 1202c between an outer perimeter portion 310o of the bottom 310b of the motor 310 and the recess 1205. Other seal configurations and types may be used, such as, for example, a gasket or other seal.

The housing 1200h can comprise an enclosure leg 1212 that couples to the enclosure cap 1202. The enclosure leg 1212 can be surround an open channel 1212c that extends under the enclosure cap 1202. The enclosure leg 1212 can have a top 1212t that is circular and attaches to the enclosure cap 1201 to sandwich the seal 1204 therebetween. The top 1212t can have an open medial portion 1212m. The shaft 310s of the drive motor 310 can extend a distance into the leg enclosure 1212 through the open medial portion 1212m. A second seal 1214 can reside above the shaft on the bottom 310b of the motor 310, between the bottom of the motor 310 and a motor shaft coupler 1220. The second seal 1214 has a smaller outer diameter than the (first) seal 1204.

Attachment members 1216 can attach the enclosure cap 1202 to the enclosure leg 1212. The attachment members 1216 can extend through pairs of aligned ears 1203, 1213 to attach the enclosures 1202, 1212 and seal the motor 310. At least one other attachment member 1219 (shown as three) can extend through the top 1212t of the leg enclosure into a bottom portion of the motor 310 to help affix/stabilize the motor to the housing 1200h.

The motor shaft coupler 1220 can be relatively short in length, typically having a length in a range of 5 mm to 50 mm and may be rigid. The motor shaft coupler 1220 can be non-ferromagnetic. The motor shaft coupler 1220 can be attached to a secondary drive shaft 1222 of smaller diameter than the drive shaft 310s that is directly attached to and/or provided by the motor 310. The secondary shaft 1222 can have an outer diameter that is in a range of 10-50% of an outer diameter of the primary drive shaft 310s. The secondary drive shaft 1222 can be coupled to a connector 229, shown as a drive wing. The connector 229 is sized and configured to couple to the actuator 200a, shown as a thumbwheel 200k in FIGS. 15A,16A.

The secondary drive shaft 1222 can extend a distance into a channel 229c of the connector 229. The secondary drive shaft 1222 can be internal to the motor shaft coupler 1220 and internal to the connector 229 with little to no externally exposed segment. Stated differently, the distal end 1220d of the motor shaft coupler 1220 can reside flush with or closely spaced apart from a proximal end 229p of the connector 229. In some embodiments, a longitudinally extending gap space “g” of between 0.001 mm and 10 mm, more typically between 0.01 and 8 mm, can reside between the distal end of the motor shaft coupler 1220 and the proximal end of the connector 229 with both held inside the enclosure leg 1212. The drive shaft (e.g., cable) 1222 can be configured to be more flexible the more exposed it is. Our current Thumb Wheel Extension, which also uses a drive cable to manually operate the thumb wheels at a distance, has an exposed length of about 8 mm.

The housing 1200h can include an outwardly extending strain relief arm 1220a that provides a cable path for control cable 1225. As shown, the strain relief arm 1200a as provided by matably configured arm segments 1207, 1217 of respective enclosure cap 1202 and leg enclosure 1212. The control cable 1225 can be non-ferromagnetic.

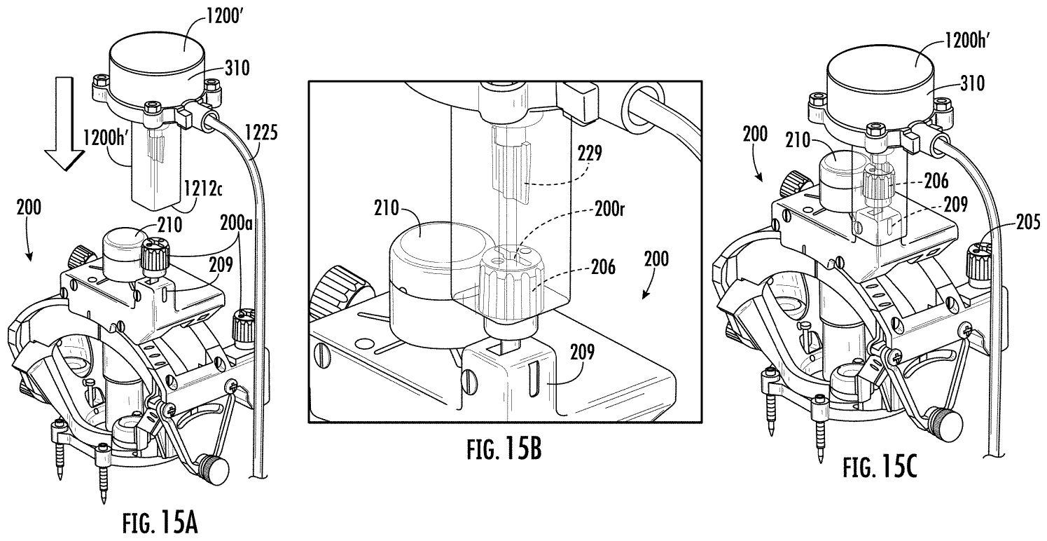

In some embodiments, a length of one side 1212s of the enclosure leg 1212 can be shorter than other sides 1212s. As shown, the side 1212s that is spaced apart 90 degrees from the side 1212s with the strain relief 1200a can have a shorter length than the other sides forming an open window region that can allow the enclosure leg 1212 to extend down over a support segment 219 under the corresponding actuator 205 (FIG. 16B, 16C) with the open/shorter side abutting or residing adjacent an arc segment of an arcuate arm 203 of the trajectory guide 200 thereby providing a lower profile assembly interface for the motor assembly 1200 and actuator 205.

The enclosure leg 1212 can have four sides 1212s₁-1212s₄ corresponding to a rectangular shape (in cross-section) as shown, but other shapes may be used such as, for example, circles, particularly where the support segment 219 has other shapes such as, for example, a cylindrical shape (not shown).

The housing 1200h can be sized and configured to slidably receive one thumbwheel 200k of a respective actuator 200a mounted on the trajectory guide 200. The thumbwheel 200k is rotatable by the connector 229 while inside an end portion of the housing 1200h. At least some of the enclosure leg 1212 may be visually translucent for ease of visualizing the attachment for ease of installation/assembly. The distal end 229d of the connector 229 can be slidably received and frictionally coupled to a (diametrically extending) slot, recess or channel 200r in an outer surface of the thumbwheel or knob 200k.

The distal end 229d of the actuator connector 229 can reside a distance “d₁” above an open distal end 1212d of the enclosure leg 1212. The distance d₁ can be in a range of about 10 mm to about 50 mm. This allows at least part, typically the entire length and width, of the actuator 200a to be received into the channel 1212c. In some embodiments, the entire thumbwheel 200k resides above the open end of the channel 1212c so that at least part of a support feature or structure 219 can also be received into the channel 1212c and provide structural support for the assembly 1200.

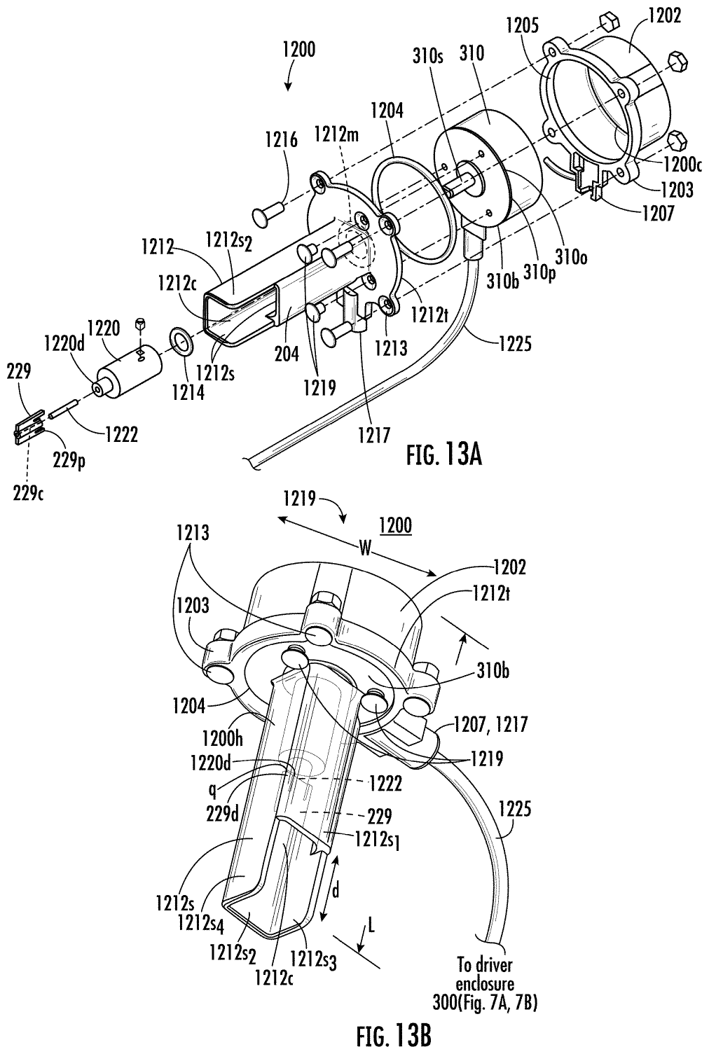

Referring to FIGS. 14A and 14B, another motor assembly 1200′ is shown. In this embodiment, the housing 1200h can comprise sides 1212s that terminate at a common distal end position and no open window is required. The enclosure leg 1212′ may have a greater maximal length than the enclosure leg 1212 shown in FIG. 13A, 13B. The enclosure leg 1212′ can be sized and configured to receive an upwardly extending (rectangular) projection 209 residing under the corresponding actuator 200a (shown as a pitch actuator 206) as shown in FIGS. 15B and 15C.

FIGS. 15A-15C and 16A-16C illustrate that each motor assembly 1200, 1200′ can be slidably coupled to a respective actuator 200a and can be concurrently supported by the trajectory guide 200. While shown as comprising only two actuators 200a, pitch and roll actuators 205, 206, the trajectory guide 200 can comprise the four actuators discussed with respect to FIG. 2 and four motor assemblies 1200, 1200′ may be coupled to the trajectory guide to drive the actuators 205, 206, 207, 208.

As shown in FIGS. 13A, 14A each actuator connector 229 of each motor assembly 1200 can be color-coded to match a color of a corresponding actuator 200a for ease in installation/assembly. Thus, a user can match colors of actuators and actuator connectors to identify actuator and actuator connector pairs (e.g., two or more of blue pairs, green pairs, yellow pairs, orange pairs), then attaching the matching color actuator and actuator connector pairs to couple the plurality of motors to the plurality of actuators.

Each control cable 1225 (FIG. 16C) can extend from the motor 310 to a housing 300 in the scanner room 100B. The housing 300 can include at least one of the controller 140, converter 133, motor drivers 145 and/or other drive control components, for example (FIGS. 1, 6, 7A, 7B). The control cable 1225 can have a length in a range of about 24 inches and 60 inches, in some embodiments.

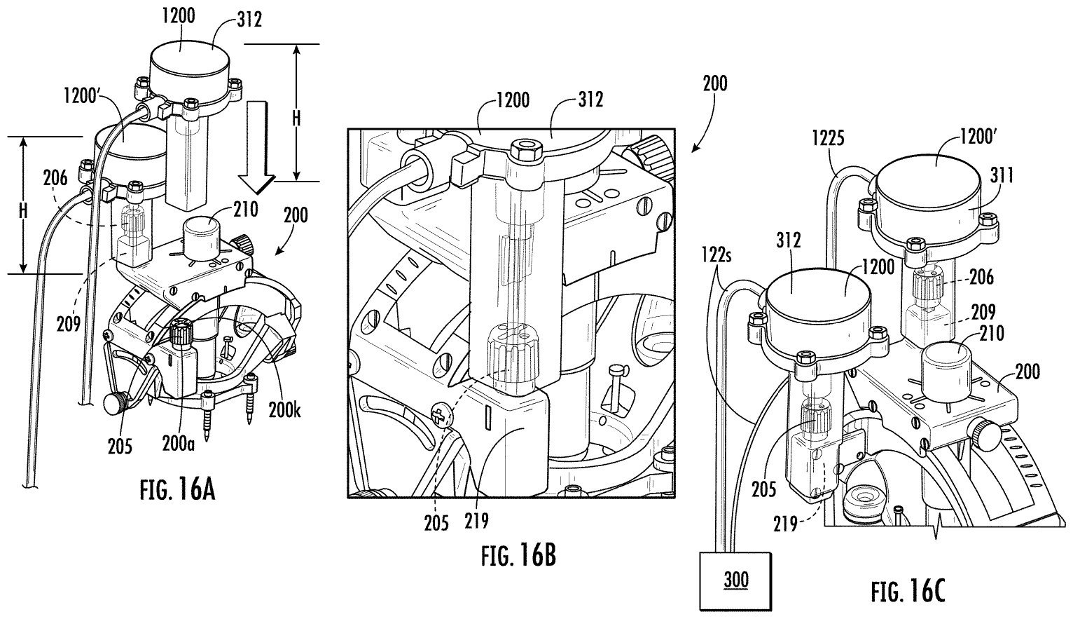

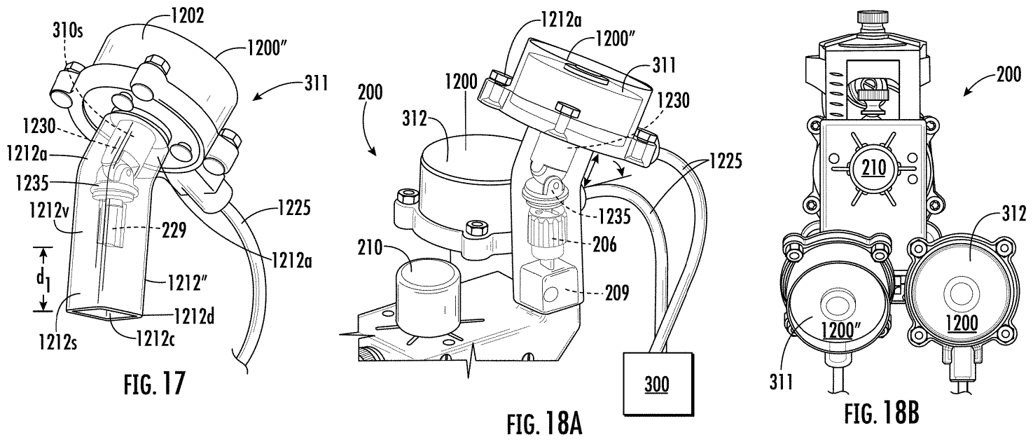

Referring to FIGS. 17, 18A and 18B, another embodiment of a motor assembly 1200″ is shown. In this embodiment the enclosure leg 1212″ can have an angled upper segment 1212a that angles from vertical in a range “β” of 30-60 degrees, under the cap enclosure 1202. The angled upper segment 1212a can merge into a vertical segment 1212v that is sized and configured to enclose the target actuator 200a (shown as pitch actuator 206). This configuration can provide additional clearance above a top stage (above the tower 210) of the trajectory guide 200. The motor assembly 1200″ can include a first coupler 1230 held inside the angled segment 1212a that couples to the shaft 310s of the motor 310 and a second coupler 1235 configured as a U-joint shaft coupler that is attached to the first coupler 1230 on one end and to the connector 229 on the other and configured to orient the connector 229 in a vertical orientation to couple with the actuator 200a, such as attach to a slot, recess or channel 200r in the actuator 200a.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

1. An image-guided surgical method, comprising:
providing an image guided surgical system comprising a computer system configured with a surgical procedure workflow;
providing a trajectory guide adapted to be held adjacent to or affixed to a patient, the trajectory guide comprising a plurality of actuators for positionally adjusting an intrabody trajectory defined by the trajectory guide for placing at least one surgical device along a desired intrabody trajectory;
providing a plurality of motors and coupling one of the plurality of motors to a corresponding one of the plurality of actuators;
providing at least one housing, wherein the least one housing contains a controller that communicates with the plurality of motors; and
transmitting instructions from the computer system of the image guided surgical system to the controller then to one or more of the plurality of motors to controllably activate one or more of the motors to turn a corresponding one or more of the plurality of actuators to adjust the intrabody trajectory defined by the trajectory guide.

2. The method of claim 1, wherein the image guided system is an MRI-image guided system coupled to an MRI scanner system, wherein the computer system resides at least partially in a control room of an MRI suite, wherein the motors are MRI-compatible motors, and wherein the at least one housing resides in a scanner room of the MRI suite.

3. The method of claim 2, wherein each MRI-compatible motor of the plurality of MRI-compatible motors is coupled to and/or comprises a control cable that extends to one or more of the at least one housing.

4. The method of claim 3, wherein the control cable has a length in a range of about 24 inches and 60 inches, and wherein the control cable and the at least one housing are formed of one or more MRI compatible materials.

5. The method of claim 1, wherein the plurality of motors are provided as a plurality of separate motor assemblies, wherein each of the motor assemblies comprise a motor assembly housing having an open channel under a sealed compartment, wherein the sealed compartment that holds a respective one of the plurality of motors, and wherein each of the motor assemblies also comprise an actuator connector held in the open channel, the method further comprising placing a respective actuator of the plurality of actuators into a corresponding open channel and attaching the actuator connector to the respective actuator.

6. The method of claim 1, further comprising matching colors of actuators and actuator connectors to identify actuator and actuator connector pairs, then attaching the matching color actuator and actuator connector pairs to couple the plurality of motors to the plurality of actuators.

7. The method of claim 1, wherein the at least one housing comprises a plurality of motor drivers that are coupled to the plurality of motors, the method further comprising controlling the plurality of motors using the motor drivers and transmitting status updates of motor operation of respective motors of the plurality of motors to the computer system of the image guided surgical system.

8. The method of claim 2, wherein the computer system of the image guided surgical system:
transmits slice plane parameters to a computer system of the MRI scanner system;
evaluates images obtained by the MRI scanner system using the slice plane parameters;
identifies a first intrabody trajectory provided by the trajectory guide as a current defined intrabody trajectory;
compares the first intrabody trajectory to the desired intrabody trajectory that intersects a target intrabody site;
calculates directional adjustments of one or more of the plurality of actuators of the trajectory guide to achieve the desired intrabody trajectory; then
transmits the instructions to a controller coupled to motor drivers that are coupled to the MRI-compatible motors; and then
directs one or more of the plurality of MRI-compatible motors to turn a corresponding actuator to adjust the first intrabody trajectory to a new defined intrabody trajectory to thereby provide the desired intrabody trajectory.

9. The method of claim 8, wherein the computer system is configured to direct two or more of the MRI-compatible motors to serially turn corresponding actuators.

10. The method of claim 8, wherein the computer system is configured to direct at least two of the MRI-compatible motors to concurrently turn at least two of the cables and corresponding actuators.

11. The method of claim 2, further comprising transmitting status data from the one or more of the plurality of MRI-compatible motors to the computer system of the image guided surgical system, wherein the method further comprises triggering the computer system of the image guided surgical system based, at least in part, on the transmitted status data, to communicate with a computer system of the MRI scanner to initiate slice acquisitions to thereby obtain further image slices.

12. The method of claim 1, wherein the plurality of actuators comprises a roll actuator and a pitch actuator.

13. The method of claim 12, wherein the plurality of actuators further comprises an X direction actuator and a Y direction actuator.

14. The method of claim 1, wherein the plurality of motors are coupled to respective rotary encoders that have a resolution in a range of about 100-300 μrad.

15. The method of claim 2, wherein the computer system of the image guided surgical system (a) transmits slice plane parameters to a computer system of the MRI scanner system to obtain images of slices extending across only a top end portion of a targeting cannula, then (b) evaluates the obtained images and identifies whether a projected error of alignment relative to the desired intrabody trajectory is within a first defined value,
wherein, if so, the computer system of the image guided surgical system then (c) transmits slice plane parameters to the computer system of the MRI scanner system to initiate acquisition of at least a first stack of images in a first plane with image slices that extend over at least a major length and/or width of the targeting cannula, and then (d) evaluates the first stack of images and identifies whether a current projected error of alignment relative to the desired intrabody trajectory is within a second defined value, wherein the second defined value is less than the first defined value.

16. The method of claim 2, wherein the MRI-compatible motors are MRI-compatible stepper motors coupled to respective encoders.

17. The method of claim 1, wherein the at least one housing further comprises a plurality of motor drivers held therein, and wherein the transmitting step comprises transmitting direction command instructions from the controller to one or more of the plurality of motor drivers that then directs an appropriate one or more of the plurality of motors to turn at a defined speed and time to carry out positional adjustments of one or more of the plurality of actuators.

18. A surgical system for an MRI suite, comprising:
a housing configured to reside in an MRI scanner room of the MRI suite, the housing comprising a controller; and
a plurality of motor assemblies including at least a first motor assembly housing and a second separate motor assembly housing, wherein the first motor assembly housing encloses a first motor and a first actuator connector, wherein the first actuator connector is coupled to the first motor, wherein the second motor assembly housing encloses a second motor and a second actuator connector, and wherein the second actuator connector is coupled to the second motor.

19. The system of claim 18, further comprising a trajectory guide comprising at least first and second spaced apart actuators, wherein the first actuator is connected to the first actuator connector of the first motor assembly and the second actuator is coupled to the second actuator connector of the second motor assembly.

20. The system of claim 19, wherein the first motor assembly housing comprises an enclosure cap coupled to an enclosure leg, wherein a motor shaft coupler and a secondary drive shaft as well as the first actuator connector and the first actuator reside in the enclosure leg of the first motor assembly housing, wherein the second motor assembly housing comprises an enclosure cap coupled to an enclosure leg, and wherein a motor shaft coupler and a secondary drive shaft as well as the second actuator connector and the second actuator reside in the enclosure leg of the second motor assembly housing.

21. The system of claim 20, wherein the first motor is sealably held in the enclosure cap above the enclosure leg of the first motor assembly housing, wherein the second motor is sealably held in the enclosure cap above the enclosure leg of the second motor assembly housing, and wherein the first motor assembly housing and the second motor assembly housing are adjacent to each other and each has a maximal longitudinal length of about 1.5 inches to about 3.5 inches.

22. The system of claim 18, wherein the first motor assembly housing comprises a first elongate cable extending outward thereof coupled at a first end portion to the first motor and coupled at a second end portion to the housing comprising the controller, wherein the second motor assembly housing comprises a second elongate cable extending outward thereof coupled at a first end portion to the second motor and coupled at an opposing second end portion to the housing comprising the controller.

23. The system of claim 22, wherein the first and second elongate cables comprise MRI compatible material or MRI compatible materials and have a length in a range of about 24 inches to about 60 inches, and wherein the first and second actuator connectors are configured to releasably couple to a respective thumbwheel of respective first and second actuators of a trajectory guide.

24. The system of claim 18, further comprising a scanner bed attachment assembly coupled to or coupleable to the housing comprising the controller.

25. The system of claim 22, wherein the housing comprising the controller, the first motor assembly housing and the second motor assembly housing are non-ferromagnetic, wherein the housing comprising the controller also contains at least one motor driver in communication with the controller and a power supply coupled to the controller and/or the at least one motor driver.

26. The system of claim 18, wherein the first and second motors are coupled to respective rotary encoders that have a resolution in a range of about 100-300 μrad.

27. The system of claim 18, wherein the housing comprising the controller comprises RF shielding.

28. The system of claim 18, wherein the housing comprising the controller is coupled to at least one fiber optic cable that has a length sufficient to extend through a wave guide of an MRI suite between a control room and a scanner room.

29. The system of claim 18, wherein the system is configured to transmit status data of the first and/or second motor to a computer system of the MR scanner system to initiate slice acquisitions to thereby obtain image slices.

30. An image-guided surgical system, comprising:
a computer system configured with a surgical procedure workflow, wherein the computer system resides at least partially in a control room of a magnetic resonance imaging (MRI) suite of a magnetic resonance (MR) scanner system;
a trajectory guide adapted to be held adjacent to or affixed to a patient, the trajectory guide comprising a plurality of actuators for positionally adjusting an intrabody trajectory defined by the trajectory guide for placing at least one surgical device along a desired intrabody trajectory; and
a plurality of motors that reside in a scanner room of the MRI suite, one motor in communication with one of the plurality of actuators of the trajectory guide, wherein the motors are MRI compatible motors,
wherein instructions from the computer system of the image guided surgical system controllably activates one or more of the motors to turn to adjust the intrabody trajectory defined by the trajectory guide.

31. The system of claim 30, further comprising a drive control enclosure comprising a controller and motor drivers, wherein the drive control enclosure resides in the scanner room, and wherein the computer system of the image guided surgical system is further configured to:
transmit slice plane parameters to a computer system of the MR scanner system;
evaluate images obtained by the MR scanner system using the slice plane parameters;
identify a first intrabody trajectory provided by the trajectory guide as a current defined intrabody trajectory;
compare the first intrabody trajectory to the desired intrabody trajectory that intersects a target intrabody site;
calculate directional adjustments of one or more of the actuators of the trajectory guide to achieve the desired intrabody trajectory; and then
transmit instructions to the controller in the drive control enclosure which then directs the motor drivers to communicate with one or more of the motors to turn a corresponding actuator to adjust the first intrabody trajectory to a new defined intrabody trajectory.

32-37. (canceled)

38. A surgical motor assembly for an image-guided surgical navigation system, comprising:
a motor assembly housing:
a motor held in the housing; and
an actuator connector held in the housing below the motor, wherein the actuator connector is coupled to the motor.

39. The assembly of claim 38, wherein the motor assembly housing comprises an enclosure cap coupled to an enclosure leg, wherein the actuator connector resides in the enclosure leg, and wherein the leg has at least one wall that surround an open downwardly extending channel.

40-47. (canceled)
